SEQUENCE LISTING (40 sequences)

SEQ ID NO: 1 - protein, 14 aa, Artificial
  Synthetic construct
KKTCRRINYCALNK

SEQ ID NO: 2 - protein, 14 aa, Artificial
  Synthetic construct
KKTLKKLWKKKKRK

SEQ ID NO: 3 - protein, 11 aa, Artificial
  Synthetic construct
RRRHQRKYRRY

SEQ ID NO: 4 - protein, 12 aa, Artificial
  Synthetic construct
FLRRRRRFTRQT

SEQ ID NO: 5 - protein, 11 aa, Artificial
  Synthetic construct
KKLALYLLLAL

SEQ ID NO: 6 - protein, 14 aa, Artificial
  Synthetic construct
KPSARMLLLKGFGK

SEQ ID NO: 7 - protein, 34 aa, Artificial
  Synthetic construct
CLKGFTLNPDKKTCRRINYCALNKPGCEHECVNM

SEQ ID NO: 8 - protein, 34 aa, Artificial
  Synthetic construct
KSSKYKAYKKKKTLKKLWKKKKRKFTPGKYFSKK

SEQ ID NO: 9 - protein, 31 aa, Artificial
  Synthetic construct
SRRSKSRSRSRRRHQRKYRRYSRSYSRSRSR

SEQ ID NO: 10 - protein, 32 aa, Artificial
  Synthetic construct
DCHDMFEHGSFLRRRRRFTRQTGAEGTRGPAK

SEQ ID NO: 11 - protein, 31 aa, Artificial
  Synthetic construct
EAPEMPALYEKKLALYLLLALSLASVAMCRT

SEQ ID NO: 12 - protein, 34 aa, Artificial
  Synthetic construct
MCLATCTRDGKPSARMLLLKGFGKDGFRFFTNFE

SEQ ID NO: 13 - protein, 956 aa, Homo sapiens [sequence omitted]

SEQ ID NO: 14 - protein, 489 aa, Artificial
  Synthetic construct
MATKKMRISEKLWDSLEKDECNIDEVVQLMSLDEEELITKFAQEVSLRIRYLDEKPNEISFIAEATEDYSEPETESSDEETYFQQIRMERGESSETKREQQDLGATRKRKIEERNPFYTPPVHKGIPSSTGRGTEISTLNLDCISSFEERKVMIDKWFNEISLIIQTNKESFDTSLKVLTLMEHRTEGIAKSFIKQATWDILITPEKIIEEVLTGFYTMFIGLDYALSAEKEEEKRIKKAEELLIKSQLCNICELDNFTCFYEKQINQLKFEDFPKWIELYLGKIPIIGKQSKERWDNEKSFTTKYSLAFAKRIIQEEIAKYCDFQRTSKKLKNFSKKCCSKNSLDPLVSFGCRDTKKKDFKKSSKYKAYKKKKTLKKLWKKKKRKFTPGKYFSKKKPEKFCPQGRKKCRCWICTEEGHYANECPNRKSHQEKVKILIHGMNEGYYPLEDAYTGNLEVFSMEIIEETTSEEESTTDSDSSSSDDEQLSF

SEQ ID NO: 15 - protein, 290 aa, Homo sapiens
MSNYVNDMWPGSPQEKDSPSTSRSGGSSRLSSRSRSRSFSRSSRSHSRVSSRFSSRSRRSKSRSRSRRRHQRKYRRYSRSYSRSRSRSRSRRYRERRYGFTRRYYRSPSRYRSRSRSRSRSRGRSYCGRAYAIARGQRYYGFGRTVYPEEHSRWRDRSRTRSRSRTPFRLSEKDRMELLEIAKTNAAKALGTTNIDLPASLRTVPSAKETSRGIGVSSNGAKPELSEKVTEDGTRNPNEKPTQQRSIAFSSNNSVAKPIQKSAKAATEEASSRSPKIDQKKSPYGLWIPI

SEQ ID NO: 16 - protein, 330 aa, Artificial
  Synthetic construct
MQQQPLPGPGAPTTEPTKPPYSYIALIAMAIQSSPGQRATLSGIYRYIMGRFAFYRHNRPGWQNSIRHNLSLNECFVKVPRDDRKPGKGSYWTLDPDCHDMFEHGSFLRRRRRFTRQTGAEGTRGPAKARRGPLRATSQDPGVPNATTGRQCSFPPELPDPKGLSFGGLVGAMPASMCPATTDGRPRPPMEPKEISTPKPACPGELPVATSSSSCPAFGFPAGFSEAESFNKAPTPVLSPESGIGSSYQCRLQALNFCMGADPGLEHLLASAAPSPAPPTPPGSLRAPLPLPTDHKEPWVAGGFPVQGGSGYPLGLTPCLYRTPGMFFFE

SEQ ID NO: 17 - protein, 3411 aa, Artificial
  Synthetic construct
MSGRKAQGKTLGVNMVRRGVRSLSNKIKQKTKQIGNRPGPSRGVQGFIFFFLFNILTGKKITAQLKRLWKMLDPRQGLAALRKVKRVVAGLMRGLSSRKRRSHDVLTVQFLILGMLLMTGGVTLMRKNRWLLLNVTSEDLGKTFSIGTGNCTTNILEAKYWCPDSMEYNCPNLSPREEPDDIDCWCYGVENVRVTYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGVADRDFIEGVHGGTWVSATLEQDKCVTVMAPDKPSLDISLETVAIDGPVEARKVCYNAVLTHVKIDDKCPSTGEAHLAEENEGDNACKRTYSDRGWGNGCGLFGKGSIVACAKFTCAKSMSLFEVDQTKIQYVIKAQLHVGAKQEDWKTDIKTLKFDVLSGSQEAEFTGYGKVTLECQVQTAVDFGNSYIAEMEKESWIVDRQWAQDLTLPWQSGSGGVWREMHHLVEFEPPHAATIRVLALGDQEGSLKTALTGAMRVTKDTNDNNLYKLHGGHVSCRVKLSALTLKGTSYKMCTDKMSFVKNPTDTGHGTVVMQVKVPKGAPCKIPVIVADDLTAAINKGILVTVNPIASTNDDEVLIEVNPPFGDSYIIIGTGDSRLTYQWHKEGSSIGKLFTQTMKGAERLAVMGDAAWDFSSAGGFFTSVGKGIHTVFGSAFQGLFGGLNWITKVIMGAVLIWVGFNTRNMTMSMSMILVGVIMMFLSLGVGADQGCAINFGKRELKCGDGIFIFRDSDDWLNKYSYYPEDPVKLASIVKASFEEGKCGLNSVDSLEHEMWRSRADEINAILEENEVDISVVVQDPKNVYQRGTHPFSRIRDGLQYGWKTWGKNLVFSPGRKNGSFIIDGKSRKECPFSNRVWNSFQIEEFGTGVFTTRVYMDAVFEYTIDCDGSILGAAVNGKKSAHGSPTFWMGSHEVNGTWMIHTLEALDYKECEWPLTHTIGTSVEESEMFMPRSIGGPVSSHNHIPGYKVQTNGPWMQVPLEVKREACPGTSVIIDGNCDGRGKSTRSTTDSGKIIPEWCCRSCTMPPVSFHGSDGCWYPMEIRPMKTHESHLVRSWVTAGEIHAVPFGLVSMMIAMEVVLRKRQGPKQVLVGGVVLLGAMLVGQVTLLDLLELTVAVGLHFHEMNNGGDAMYMALIAAFSVRPGLLIGFGLRTLWSPRERLVLALGAAMVEIALGGMMGGLWKYLNAVSLCILTINAVASRKASNAILPLMALLTPVTMAEVRLATMLFCTVVIIGVLHQNSKDTSMQKTIPLVALTLTSYLGLTQPFLGLCAFLATRIFGRRSIPVNEALAATGLVGVLAGLAFQEMENFLGPIAVGGILMMLVSVAGRVDGLELKKLGEVSWEEEAEISGSSARYDVALSEQGEFKLLSEEKVPWDQVVMTSLALVGAAIHPFALLLVLAGWLFHVRRARRSGDVLWDIPTPKIIEECEHLEDGIYGIFQSTFLGASQRGVGVAQGGVFHTMWHVTRGAFLVWNGKKLIPSWASVKEDLVAYGGSWKLEGRWDGEEEVQLIAAAPGKNVVNVQTKPSLFKVRNGGEIGAVALDYPSGTSGSPIVNRNGEVIGLYGNGILVGDNSFVSAISQTEVKEEGKEELQEIPTMLKKGKTTILDFHPGAGKTRRFLPQILAECARRRLRTLVLAPTRVVLSEMKEAFHGLDVKFHTQAFSAHGSGREVIDVMCHATLTYRMLEPTRIVNWEVIIMDEAHFLDPASIAARGWAAHRARANESATILMTATPPGTSDEFPHSNGEIEDVQTDIPSEPWNTGHDWILADKRPTAWFLPSIRAANVMAASLRKAGKSVVVLNRKTFEREYPTIKQKKPDFILATDIAEMGANLCVERVLDCRTAFKPVLVDEGRKVAIKGPLRISASSAAQRRGRIGRNPNRDGDSYYYSEPTSEDNAHHVCWLEASMLLDNMEVRGGMVAPLYGVEGIKTPVSPGEMRLRDDQRKVFRELVRNCDLPVWLSWQVAKAGLKTNDRKWCFEGPEEHEILNDSGETVKCRAPGGAKKPLRPRWCDERVSSDQSALSEFIKFAEGRRGAADVLVVLSELPDFLAKKGGEAMDTISVFLHSEEGSRAYRNALSMMPEAMTIVMLFLLAGLLTSGMVIFFMSPKGISRMSMAKGTMAGCGYLMFLGGVEPTHISYIMLIFFVLMVVVIPEPGQQRSIQDNQVAFLIIGILTLVSVVAANELGMLEKTKEDLFGKKNLIPSGASPWSWPDLDLKPGAAWTVYVGIVTMLSPMLHHWIKVEYGNLSLSGIAQSASVLSFMDKGIPFMKMNISVIMLLVSGWNSITVMPLLCGIGCAMLHWSLILPGIKAQQSKLAQRRVFHGVAKNPVVDGNPTVDIEEAPEMPALYEKKLALYLLLALSLASVAMCRTPFSLDEGIVLASAALGPLIEGNTSLLWNGPMAVSMTGVMRGNYYAFVGVAYNLWKMKTARRGTANGKTLGEVWKRELNLLDKQQFELYKRTDIVEVDRDTARRHLAEGKVDTGVAVSRGTAKLRWFHERGYVKLEGRVIDLGCGRGGWCYYAAAQKEVSGVKGFTLGRDGHEKPMNVQSLGWNIITFKDKTDVHPLEPVKCDTLLCDIGESSSSSVTEGERTVRVLDTVEKWLACGVDNFCVKVLAPYMRDVLEKLELLQRRFGGTVIRNPLSRNSTHEMYYVSGARSNVTFTVNQTSRLLMRRMRRPTGKVTLEADVTLPIGTRSVETDKGPLDKEAIKERVERIKSEYMTSWFYDNDNPYRTWHYCGSYVTKTSGSAASMVNGVIKLLTYPWDKIEEVTRMAMTDTTPFGQQRVFKEKVDTRAKDPPAGTRKIMKVVNRWLFRHLAREKNPRLCTKEEFIAKVRSHAAIGAYLEEQDQWKTANEAVQDPKFWELVDEERKLHQQGRCRTCVYNMMGKREKKLSEFGKAKGSRAIWYMWLGARYLEFEALGFLNEDHWASRENSGGGVEGIGLQYLGYVIRDLAAMDGGGLYADDTAGWDTRITEADLDDEQEILNYMSPHHKKLAQAVMEMTYKNKVVKVLRPAPGGKAYMDVISRRDQRGSGQVVTYALNTITNLKVQLIRMAEAEMVIHHQHVQDCDESVLTRLEAWLTEHGCDRLRRMAVSGDDCVVRPIDDRFGLALSHLNAMSKVRKDISEWQPSKGWNDWENVPFCSHHFHELQLKDGRRIVVPCREQDELIGRGRVSPGNGWMIKETACLSKAYANMWSLMYFHKRDMRLLSLAVSSAVPTSWVPQGRTTWSIHGKGEWMTTEDRLEVWNRVWITNNPHMQDKTMVKEWRDVPYLTKRQDKLCGSLIGMTNRATWASNIHLVIHRIRTLVGQEKYTDYLTVMDRYSVDADLQPGELI

SEQ ID NO: 18 - protein, 261 aa, Homo sapiens
MTCWLRGVTATFGRPAEWPGYLSHLCGRSAAMDLGPMRKSYRGDREAFEETHLTSLDPVKQFAAWFEEAVQCPDIGEANAMCLATCTRDGKPSARMLLLKGFGKDGFRFFTNFESRKGKELDSNPFASLVFYWEPLNRQVRVEGPVKKLPEEEAECYFHSRPKSSQIGAVVSHQSSVIPDREYLRKKNEELEQLYQDQEVPKPKSWGGYVLYPQVMEFWQGQTNRLHDRIVFRRGLPTGDSPLGPMTHRGEEDWLYERLAP

SEQ ID NO: 19 - protein, 11 aa, Homo sapiens
YGRKKRRQRRR

SEQ ID NO: 20 - protein, 16 aa, Artificial
  Synthetic construct
RQIKIWFQNRRMKWKK

SEQ ID NO: 21 - protein, 9 aa, Artificial
  Synthetic construct
RRRRRRRRR

SEQ ID NO: 22 - protein, 13 aa, Artificial
  Synthetic construct
VATIKSVSFYTRK

SEQ ID NO: 23 - protein, 9 aa, Artificial
  Synthetic construct
RPPGFSPFR

SEQ ID NO: 24 - protein, 29 aa, Artificial
  Synthetic construct
VATIKSVSFYTRKGGKLAKLAKKLAKLAK

SEQ ID NO: 25 - protein, 25 aa, Artificial
  Synthetic construct
RRRRRRRRRGGKLAKLAKKLAKLAK

SEQ ID NO: 26 - protein, 27 aa, Artificial
  Synthetic construct
YGRKKRRQRRRGGKLAKLAKKLAKLAK

SEQ ID NO: 27 - protein, 30 aa, Artificial
  Synthetic construct
KKTLKKLWKKKKRKGGKLAKLAKKLAKLAK

SEQ ID NO: 28 - protein, 30 aa, Artificial
  Synthetic construct
KKTCRRINYCALNKGGKLAKLAKKLAKLAK

SEQ ID NO: 29 - protein, 27 aa, Artificial
  Synthetic construct
RRRHQRKYRRYGGKLAKLAKKLAKLAK

SEQ ID NO: 30 - protein, 30 aa, Artificial
  Synthetic construct
KPSARMLLLKGFGKGGKLAKLAKKLAKLAK

SEQ ID NO: 31 - protein, 14 aa, Artificial
  Synthetic construct
KLAKLAKKLAKLAK

SEQ ID NO: 32 - protein, 28 aa, Artificial
  Synthetic construct
FLRRRRRFTRQTGGKLAKLAKKLAKLAK

SEQ ID NO: 33 - protein, 50 aa, Artificial
  Synthetic construct
PLVSFGCRDTKKKDFKKSSKYKAYKKKKTLKKLWKKKKRKFTPGKYFSKK

SEQ ID NO: 34 - DNA, 42 bp, Artificial
  Oligonucleotide
aagaaaacctgcagaaggatcaactactgtgcactgaacaaa

SEQ ID NO: 35 - DNA, 42 bp, Artificial
  Oligonucleotide
aagaaaacccttaaaaaactctggaagaaaaagaaaagaaaa

SEQ ID NO: 36 - DNA, 33 bp, Artificial
  Oligonucleotide
cgaaggcgccaccagcggaagtacaggcgctac

SEQ ID NO: 37 - DNA, 36 bp, Artificial
  Oligonucleotide
ttcctacgccgccgccgccgcttcacccggcagaca

SEQ ID NO: 38 - DNA, 33 bp, Artificial
  Oligonucleotide
aagaaactggctctatatctccttcttgctctc

SEQ ID NO: 39 - DNA, 42 bp, Artificial
  Oligonucleotide
aaaccctctgctcgcatgttgctgctgaagggcttcgggaaa

SEQ ID NO: 40 - protein, 10 aa, Artificial
  Synthetic construct
RRRRRRRRRK

(19) United States
(12) Patent Application Publication
(10) Pub. No.: US 2014/0056811 A1
(43) Pub. Date: Feb. 27, 2014

(54) NEW CELL-PENETRATING PEPTIDES AND USES THEREOF

(71) Applicants: Etai Jacob, Qiryat Ono (IL); Amir Toporik, Holon (IL); Ronen Shemesh, Modiin (IL); Ofer Levy, Moshav Mesisraelat Zion (IL)

(72) Inventors: Etai Jacob, Qiryat Ono (IL); Amir Toporik, Holon (IL); Ronen Shemesh, Modiin (IL); Ofer Levy, Moshav Mesisraelat Zion (IL)

(73) Assignee: COMPUGEN LTD., Tel Aviv-Yafo (IL)

(21) Appl. No.: 13/976,502

(22) PCT Filed: Dec. 26, 2011
(86) PCT No.: PCT/IB2011/055956

§ 371 Date: Nov. 5, 2013

Related U.S. Application Data
(60) Provisional application No. 61/427,208, filed on Dec. 27, 2010.

(51) Int. Cl. A61K 47/48

(52) U.S. Cl. CPC A61K 47/48246
USPC 424/1.69; 530/327; 530/317; 530/322; 530/350; 424/193.1; 514/19.3; 424/9.1; 530/324

(57) ABSTRACT

The present invention is related to peptides, which are suitable for use as cell-penetrating peptides (CPPs), variants thereof and/or complexes, fusion molecules and/or conjugates comprising same, use thereof for manufacture of compositions for diagnosing, treating and/or preventing of medical conditions.

### FIELD OF THE INVENTION

The present invention is related to peptides, which are suitable for use as cell-penetrating peptides (CPPs), and also relates to complexes, fusion molecules and/or conjugates comprising same, and uses thereof for treating, preventing, diagnosing and/or rendering a prognosis for medical conditions.

### BACKGROUND OF THE INVENTION

A number of techniques have been developed to deliver different cellular effectors into cells. The majority of these techniques are invasive, like electroporation or microinjection. Liposome encapsulation and receptor-mediated endocytosis are milder methods, but they unfortunately suffer from serious drawbacks, in particular, low delivery yield.

The established view in cellular biology dictates that the cellular internalization of hydrophilic macromolecules can only be achieved through the classical endocytosis pathway. However, in the last decade, several peptides have been demonstrated to translocate across the plasma membrane of eukaryotic cells by a seemingly energy-independent pathway. These peptides are defined as cell-penetrating peptides (CPPs) and have been used successfully for intracellular delivery of macromolecules with molecular weights several times greater than their own. (M. Undgren et al, 2000, Cell-penetrating peptides; TIPS, Vol. 21, pg. 99-103)

Cellular delivery using these cell-penetrating peptides offers several advantages over conventional techniques. It is non-invasive, energy-independent, is efficient for a broad range of cell types and can be applied to cells en masse. Furthermore, it has been found that for certain types of CPPs, cellular internalisation occurs at 37° C., as well as at 4° C., and that it can not be saturated.

Cell-penetrating peptides (CPPs) such as the antennapedia-derived penetratin (Derossi et al., Biol. Chem., 269, 10444-10450, 1994) and the Tat peptide (Vives et al., J. Biol. Chem., 272, 16010-16017, 1997) are widely used tools for the delivery of cargo molecules such as peptides, proteins and oligonucleotides (Fischer et al., Bioconjug. Chem., 12, 825-841, 2001) into cells. Areas of application range from purely cell biological to biomedical research (Dietz and Bähr, Mol. Cell., Neurosci, 27, 85-131, 2004). Initially, cellular uptake was believed to occur by direct permeation of the plasma membrane (Prochiantz, Curr. Opin. Cell Biol., 12, 400-406, 2000). Recently, evidence has accumulated that for several CPPs, endocytosis is a cellular uptake mechanism (for a review, see Fotin-Mleczek et al., Curr. Pharm. Design, 11, 3613-3628, 2005). Given these recent results, the functional behavior of a peptide as a CPP therefore does not imply a specific cellular import mechanism, but rather refers to a peptide that by itself penetrates the cellular membrane, or that when attached to a cargo, either covalently or non-covalently, enhances the cellular uptake of the cargo molecule.

Although these CPPs have been proven to be potentially suitable for the delivery of peptides, proteins, oligonucleotides, nano-particles and any bioactive molecule into cells, they all suffer from limitations, such as their effectiveness being restricted to a subset of cargo molecules. Other peptides are relatively inefficient in the delivery of cargo molecules or have at least some specificity for delivery to various tissues (Mueller et al., Bioconjugate Chem., 2008, 19 (12), pp 2363-2374; El-Andaloussi et al., Biochem J. 2007 October 15; 407(Pt 2): 285-292).

### SUMMARY OF THE INVENTION

There is a need for, and it would be useful to have, CPPs which have one or more of the following characteristics (without wishing to be limited by a closed list): (i) the CPP supports rapid release of cargo molecule from the endolysomal pathway; (ii) the CPP does not cause immunological reactions in humans; (iii) the CPP is useful for a wide variety of bioactive substances and hence is potentially useful as a delivery platform for therapeutic drugs and/or prophylactic drugs for a wide variety of diseases; (iv) the CPP provides target specificity; (v) the CPP acts as a diagnostic tool to visualize internal features and physiological conditions, for example by combining CPPs with imaging labels in an effort to optimize aspects of in vivo imaging; and (vi) the CPP crosses the blood brain barrier with its cargo molecule.

According to at least some embodiments the present invention provides a peptide having an amino acid sequence selected from any one of SEQ ID NOs: 1-6, variants, complexes, fusions and conjugates thereof, having an activity of a cell-penetrating peptide (collectively referred herein as “CPP(s)”). The term “variant” comprises any suitable variation or change to the peptide from the listed amino acid sequences. Without wishing to be limited in any way, such CPPs may optionally have therapeutic and/or diagnostic value for a wide range of conditions, disorders and diseases. Optionally, according to at least some embodiments, “variants” may comprise fragments, homologs, and derivatives of the peptides.

By “homolog” it is meant that the peptide has at least 90% homology and preferably at least 95% homology; and/or that any substitutions are conservative amino acid substitutions as described below.

By “fragment” it is meant that the peptide contains at least 60%, preferably at least 70%, more preferably at least 80% and most preferably at least 90% of an amino acid sequence as given herein.

By “derivative” it is meant that the peptide has been derivatized in some manner, for example through the addition of one or more chemical moieties, to the basic structure of the peptide itself.

Preferably, peptides according to at least some embodiments of the invention have a length of 5 to 40 amino acids, more preferably of 6 to 30 amino acids, more preferably of 6 to 22, more preferably of 6 to 21, more preferably of 6 to 20, and most preferably 8-19 amino acids.

In at least some embodiments, the subject invention thus provides a peptide consisting essentially of an amino acid sequence KKTCRRINYCALNK (S22 [SEQ ID NO: 1]) or a variant thereof.

In at least some embodiments, the subject invention thus provides a peptide consisting essentially of an amino acid sequence KKTLKKLWKKKKRK (S24 [SEQ ID NO: 2]) or a variant thereof.

In at least some embodiments, the subject invention thus provides a peptide consisting essentially of an amino acid sequence RRRHQRKYRRY (S36 [SEQ ID NO: 3]) or a variant thereof.

In at least some embodiments, the subject invention thus provides a peptide consisting essentially of an amino acid sequence FLRRRRRFTRQT (S40 [SEQ ID NO: 4]) or a variant thereof.

In at least some embodiments, the subject invention thus provides a peptide consisting essentially of an amino acid sequence KKLALYLLLAL (S67 [SEQ ID NO: 5]) or a variant thereof.

In at least some embodiments, the subject invention thus provides a peptide consisting essentially of an amino acid sequence KPSARMLLLKGFGK (S8 [SEQ ID NO: 6]) or a homolog, fragment or a derivative thereof.

In at least some embodiments, some non-limiting illustrative examples of variants of SEQ ID NOs:1-6 are depicted in SEQ ID NOs:7-12, respectively. Another non-limiting illustrative example of a variant of SEQ ID NO:2 according to at least some embodiments of the invention is SEQ ID NO:33.

According to at least some embodiments but without wishing to provide a closed list, the present invention provides complexes, fusions and/or conjugates of the CPPs, wherein the peptide is preferably attached to another entity through any one of the following:
1. Conjugation chemistry (optionally through derivatization of one or both of the peptide and/or the cargo) and hence formation of a covalent bond:
   a. Conjugates through Carboxyl groups.
   b. Conjugates through free amines
   c. Conjugates through the thiol group on cysteine
   d. Conjugates through one or more added moieties for derivatization of one or both of the peptide and/or the cargo, including optionally through any of the groups of a-c.
2. Non covalent interactions between the CPP and another entity/ies, and hence formation of a non-covalent bond:
a. Complexation—solution of CPP(s) and molecular entity in different proportions.
b. Complexation as (a) with additional carrier proteins such as Keyhole limpet hemocyanin (KLH), bovine serum albumin (BSA), ovalbumin (OVA) or Rabbit Serum Albumin (RSA).

With regard to the above non-covalent bond, optionally such a non-covalent bond may comprise one or more of ionic bonds, hydrogen bonds or hydrophobic interaction or a combination of such bonds.

According to at least some embodiments of the present invention, such a covalent bond may optionally be formed through any chemical groups, polymers, peptides, proteins or other linkers known in the art. Such linkers may be chosen to be composed of flexible residues like Glycine and Serine so that the adjacent entities (e.g. CPP and cargo) are free to move relative to one another or such that the inherent biological or therapeutic function of the cargo and CPP must remain or improved or at least should not be worsen after modification and conjugation (as the flexible linker Gly-Gly used in Example 3 herein, and described in Watkins., et al., Journal of Controlled Release 2009, 140:237-244, to conjugate between the pro-apoptotic peptide and a CPP or as Valine-citruline (Val-Cit) pairs which may be linked to cargos from the family of drugs such as monomethyl auristatin E (MMAE)). Alternatively, such linkers may be chosen to be selective cleavable (e.g. by enzyme-cleavable) as disulfide-based linkers; where the bond between two Cysteine amino acids is reduced in the Cytosol or Hydrazone linkers; which have been designed to be selectively cleaved within the intracellular compartment of lysosomes (lower pH compared to the systemic blood circulation). Thus, linkers are preferably selected from the group consisting of any one of: (Gly-Ser)ₙ, (Gly-Gly-Ser-Gly)ₙhydrophobic linker (e.g. poly leucine), polar linker (as Poly Serine), neutral linker (as poly Glycine, poly Histidine or Lysine), e.g. in order to associate a protein or protein domain or nano-sized particle, DNA/RNA binding domain, e.g. in order to associate CPPs with therapeutic RNAi moiety, carbohydrates, amphipathic and lipophilic molecules.

According to further embodiments, the peptide further comprises a moiety which is suitable for any detection scheme or tracing methodology for in vivo or in vitro constellation, using a fluorescent reading instrument including but not limited to fluorescence microscope, flow cytometer or magnetic resonance imaging (MRI) device, whereby such moiety is preferably biologically non reactive and selected from the group comprising fluorophores, radioactive tracers, magnetic labeling, haptens and biotin such that the latter for example, in its simplest form, avidin-biotin detection methods entail applying a biotinylated probe to a sample and then detecting the bound probe with a labeled avidin or streptavidin.

According to further embodiments, the CPP peptide is radioactively labeled, preferably by having incorporated a radioactively labeled amino acid.

According to at least some embodiments the present invention provides a complex, a fusion, and/or a conjugate, comprising a peptide selected from the CPPs attached to a cargo molecule.

According to further embodiments the cargo molecule is covalently or non-covalently attached to the CPP.

According to still further embodiments the cargo molecule is selected from the group consisting of nucleic acids, amino acids, peptides, proteins, carbohydrates, lipids, and small molecules and mixtures of any of thereof.

According to still further embodiments the cargo molecule is present in a structure or part of a structure, whereby the structure is selected from the group comprising nanoparticles, microparticles, liposomes, carbon nano-tubes and micelles.

According to still further embodiments the nucleic acid cargo molecule comprises a nucleic acid selected from the group comprising DNA molecules, RNA molecules, PNA molecules, siRNA molecules, miRNA molecules, antisense molecules, ribozymes, aptamers, spiegelmers and decoy molecules.

According to still further embodiments the peptide cargo molecule comprises a peptide having therapeutic activity and in a more preferred embodiment a therapeutic activity to treat a disease.

According to still further embodiments the peptide cargo molecule is selected from the group comprising peptides for vaccination.

According to still further embodiments the nucleic acid cargo molecule is a nucleic acid-based vaccine.

According to still further embodiments the nano-particles and/or the micro-particles comprise or consist of a pharmaceutically active compound.

According to at least some embodiments the present invention provides a composition comprising at least one CPP and optionally at least one separate cargo molecule. According to some embodiments the at least one CPP provides the function of the cargo molecule and so no cargo molecule or other therapeutically active molecule is present; for these embodiments, preferably the composition comprises a pharmaceutically suitable carrier. As used herein, the term “separate” does not necessarily mean that the cargo molecule is not linked, covalently or non-covalently, to the CPP.

According to further embodiments the present invention provides a composition comprising a complex as described above.

According to further embodiments the present invention provides a composition comprising a fusion and/or a conjugate as described above.

According to at least some embodiments the present invention provides a nucleic acid coding for any one of the CPP peptides. According to further embodiment the nucleic acid coding for any one of the CPPs has a nucleic acid sequence according to SEQ ID NOs: 34-39, or a degenerative variants thereof.

According to at least some embodiments the present invention provides a composition comprising a nucleic acid coding for any one of the CPPs and a cargo molecule.

According to further embodiments the cargo molecule is a nucleic acid preferably with a therapeutic effect, such as antisense molecule, PNA, siRNA molecule or an miRNA molecule.

According to still further embodiments the cargo molecule is a nucleic acid coding for a peptide such as for example, therapeutic peptide, ligand, enzyme, recombinant proteins, and so on.

According to still further embodiments the nucleic acid coding for the CPP peptide is covalently linked to, or otherwise provided with, the nucleic acid coding for another, therapeutic peptide, such that the CPP and the therapeutic peptide would be co-expressed in the same cell.

According to still further embodiments the nucleic acid coding for the CPP peptide and the nucleic acid coding for a peptide are linked in-frame.

According to still further embodiments the peptide is a pharmaceutically active agent.

According to at least some embodiments the present invention provides use of a CPP as a cell-penetrating peptide.

According to at least some embodiments the present invention provides use of CPP peptide as a transfection agent.

According to at least some embodiments the present invention provides use of a composition according to at least some embodiments of the present invention for the manufacture of a medicament.

According to further embodiments the cargo molecule is a pharmaceutically active agent.

According to at least some embodiments the present invention provides use of a composition according to at least some embodiments of the present invention for the manufacture of a diagnostic agent.

According to further embodiments the cargo molecule is a drug or disease related marker, such as diagnostic marker to diagnose an existing disease, and/or a prognostic marker to detect if such a disease may develop. Preferably, such markers can enable the detection of the levels and activities of specific targets and more preferably, such markers can enable the detection of the levels and activities of specific intracellular targets.

According to at least some embodiments of the present invention provides a CPP-complex, fusion and/or conjugate. According to further embodiments, the CPP-complex, fusion and/or conjugate is designed so that the cell penetration-active structure of the CPPs is modified by a covalently or non-covalently attached entity in order to produce specificity to a certain malignant tissue, process or other condition such that the modifying entity is disconnected or being deactivated in proximity to the tissue, process or condition followed by an activation of the CPP penetration ability and by such a progression of events, result in a therapeutic effect. Preferably such an entity is a molecule with certain affinity to the CPP, such as small molecule, lipid like molecule, oligonucleotide or a polymer construct and more preferably a peptide.

According to at least some embodiments, the present invention relates to cell type or tissue specific targeted CPPs. According to further embodiments, the cell type or tissue specific targeted CPPs are further modified by a non-covalent intermolecular interaction or a covalent conjugation with a targeting entity which can selectively target certain tissue types, including but not limited to malignant or otherwise pathological tissue, sparing normal tissues or target a specific organ. Such targeting entity may be a part of a receptor targeting sequence, or targeting peptide (homing peptide) such as those peptides discovered by phage display for example as further described below, or any other technique or method which produces molecules capable of targeting the CPP (and optionally any cargo molecule) to a specific cell, cell type and/or tissue.

According to at least some embodiments the present invention provides a composition comprising such a targeting entity-CPP construct and a cargo molecule of any type mentioned above.

### BRIEF DESCRIPTION OF THE FIGURES

FIG. 1 presents FACS analysis results of quantification of cellular uptake of control peptides, N5 (SEQ ID NO:23) and Penetratin (SEQ ID NO:20) at 37° C. in different doses. The X axis represents the fluorescent intensity measured and the Y axis represents the number of cells with the intensity measured. The different lines indicate different micromolar amounts of the respective peptides as shown on the Figure.

FIG. 2 presents FACS analysis results of quantification of cellular uptake of FAM labeled S24 (SEQ ID NO:1) peptide in different doses. The X axis represents the fluorescent intensity measured and the Y axis represents the number of cells with the intensity measured. The different lines indicate different micromolar amounts of the specific peptide S24 as shown on the Figure.

FIG. 3 presents single section confocal microscopy images divided into four quarters A-D where each quarter represents the cellular distribution of the peptide at concentration of 10 uM at 37° C., showing the accumulation and distribution of peptides inside cells. FIG. 3A: TAT (SEQ ID NO: 19); FIG. 3B: Penetratin (SEQ ID NO: 20); FIG. 3C: 9R (SEQ ID NO: 21); and FIG. 3D: N5 (SEQ ID NO: 23). Each quarter of the figure, labeled A-D, presents four images, numbered 1-4 within the quarter, which are based on identical images sampled using different color filters of the confocal microscopy, as follows: sub quarter 1 (i.e. FIGS. 3A-1, 3B-1, 3C-1 and 3D-1) is based on a green color filter, which demonstrates the distribution of the FAM labeled peptides inside the cells as dark gray; sub quarter 2 (i.e. FIGS. 3A-2, 3B-2, 3C-2 and 3D-2) demonstrates the original image without any filtering; sub quarter 3 (i.e. FIGS. 3A-3, 3B-3, 3C-3 and 3D-3) is based on a red color filter, demonstrating the cells membrane staining as bright gray; and sub quarter 4 (i.e. FIGS. 3A-4, 3B-4, 3C-4 and FIG. 3D-4) demonstrates both red and green original colors as bright and dark gray.

FIG. 4 presents the equivalent single cross section images of the four control peptides described in FIG. 3, at a concentration of 10 uM at 37° C., as follows: TAT (SEQ ID NO: 19) (FIG. 4A); Penetratin (SEQ ID NO: 20) (FIG. 4B); 9R (SEQ ID NO: 21) (FIG. 4C); and N5 (SEQ ID NO: 23) (FIG. 4D); showing the accumulation and distribution of peptides inside cells (e.g. cytoplasm).

FIG. 5 presents the cellular distribution of peptide S24 (SEQ ID:2). Quarters A-D are based on identical images, sampled using different color filters of the confocal microscopy, as follows: quarter A (i.e. FIG. 5A) is based on a green color filter, which shows the distribution of the FAM labeled peptides inside the cells as dark gray; quarter B (i.e. FIG. 5B) shows the original image without any filtering; quarter C (i.e. FIG. 5C) is based on a red color filter, showing the cell membrane staining as bright gray; and quarter D (i.e. FIG. 5D) shows both red and green original colors as bright and dark gray. FIG. 5E presents a single cross section of the cellular distribution of S24 (SEQ ID:2) peptide. FIGS. 5A-E illustrate the intracellular accumulation of S24 peptide, not only its appearance on the outer cell surface (membrane), thereby confirming the efficient cellular uptake and intracellular accumulation of this peptide.

FIG. 6 presents a single cross section of confocal microscopy images demonstrating cellular distribution of FAM labeled S22 peptide (SEQ ID NO: 1), (5 uM, 37° C.). FIG. 6A presents intracellular distribution of S22 peptide (dark gray); FIG. 6B presents membrane staining (bright gray); FIG. 6C presents both S22 peptide cellular distribution and membrane staining; FIG. 6D shows the cellular distribution of S22 peptide, in single section based on a green color filter. FIG. 6A-D illustrates the intracellular accumulation of S22 peptide, not only its appearance on the outer cell surface (membrane), thereby confirming the efficient cellular uptake and intracellular accumulation of this peptide.

FIG. 7 demonstrates MTT assessed viability of Hela cells as a measurement for (klaklak)2 intracellular delivery in two different peptide concentrations: 26.6 ug/ml (dark grey bars) and 8.88 ug/ml (light grey bars). The peptides analyzed are (klaklak)2 alone (SEQ ID NO:31), and (klaklak)2 conjugated to any one of S8 (SEQ ID NO:30), S40 (SEQ ID NO:32), S24 (SEQ ID NO:27) and S36 (SEQ ID NO:29), from left to right, respectively. The Y axis represents the percentage (%) of viable cells (normalized by untreated cells and averaged over 2 independent experiments).

FIG. 8 demonstrates the MTT assessed viability results of Hela cells as a measurement for (klaklak)2 intracellular delivery, wherein (klaklak)2 peptide was conjugated to control peptides N2 (SEQ ID NO: 24) (FIG. 8A), R9 (SEQ ID NO:25) (FIG. 8B) or Tat (47-57) (SEQ ID NO:26) (FIG. 8C). The Y axis represents the % of viable Hela cells (normalized by untreated cells), thereby indicating the efficacy of (klaklak)2 peptide in killing these cells, which in turn is determined by the efficacy of the CPP in bringing the (klaklak)2 peptide into the cell. The X axis is presented as a log uM concentration. The results are an average of two independent experiments. The EC50 value (provided if it can be estimated with a high level of confidence) is the concentration in which 50% of maximum efficacy is achieved.

FIG. 9 demonstrates MTT assessed results of the viability of Hela cells as a measurement for (klaklak)2 intracellular delivery, wherein (klaklak)2 peptide was conjugated to S24 (SEQ ID NO: 27) (FIG. 9A) or to S36 (SEQ ID NO: 29) (FIG. 9B). The Y axis represents the % of viable cells normalized by untreated Hela cells of duplicated experiments thereby again indicating the efficacy of the CPP in bringing the (klaklak)2 peptide into the cell. The X axis is presented as a log uM concentration.

FIG. 10 presents flow cytometry analysis with cells of the A549 cell line with five different FAM (Carboxyfluorescein) labeled CPPs: 50 μl liposomes covered with negative control CPP N2 (SEQ ID NO: 22), positive control CPP 9R-K (SEQ ID NO: 40), CPP S22 (SEQ ID NO: 1), CPP S24 (SEQ ID NO: 2), CPP S8 (SEQ ID NO: 6) (0.5 mg/ml each), or 50 μl liposome only, in a comparison with control unstained cells.

FIG. 11 presents flow cytometry analysis with cells of the NAR cell line with five different FAM-CPPs: 50 μl liposomes covered with CPP N2 (SEQ ID NO: 22), CPP 9R-K (SEQ ID NO: 40), CPP S22 (SEQ ID NO: 1), CPP S24 (SEQ ID NO: 2), CPP S8 (SEQ ID NO: 6) (0.5 mg/ml each), or 50 μl liposome only, in a comparison with control unstained cells.

FIG. 12 shows confocal microscope (Meta 510) images demonstrating internalization of liposomes conjugated to CPP S22 into cells of the NRK cell line. The results present internalization following 1 hour of incubation at 37° C. with: 10 μM of free peptide (FIG. 12A), or 50 μl of liposome covered with CPP S22 (SEQ ID NO: 1) without EDC-NHS (FIG. 12B), or 50 μl of liposome covered with CPP S22 with EDC-NHS (FIGS. 12C and 12D). Hoechst reagent (Bisbenzimide Hoechst No. 33342 Trihydrochloride—Product No. B2261-100MG) 1:10,000 and P-cadherin antibody (1:150) were used as nuclei and membrane markers, respectively.

FIG. 13 shows confocal microscope images demonstrating internalization results of a different CPP, CPP S8 (SEQ ID NO: 6), also into cells of the NRK cell line. The results present internalization following 1 hour of incubation at 37° C. with 10 μM of free peptide (FIG. 13A), or 50 μl of liposome covered with CPP without EDC-NHS (FIG. 13B), or 50 μl of liposome covered with CPP with EDC-NHS (FIG. 13C), or 50 μl liposome only (FIG. 13D). Membrane and nuclei markers used are the same as those described in FIG. 12.

FIG. 14 shows confocal microscope images demonstrating internalization results of CPP S22 (SEQ ID NO: 1) into cells of the A549 cell line. The results present internalization following 1 hour of incubation at 37° C. with 10 μM of free peptide (FIG. 14A), 50 μl of liposome covered with CPP without EDC-NHS (FIG. 14B), or 50 μl of liposome covered with CPP with EDC-NHS (FIGS. 14C and 14D). Membrane and nuclei markers used are the same as those described in FIG. 12. For all images in this figure: labeling of a part of the image with the word “green” indicates successful penetration of a CPP alone to the cell. Labeling of a part of the image with the word “red” indicates successful internalization of a liposome and labeling of a part of the image with the word “orange” indicates successful penetration of the CPP with the liposome together. This figure resembles the specific internalization of CPPs and liposome to A549 cells.

FIG. 15 shows internalization of liposomes without CPP into cells of the A549 cell line using confocal microscope. The results present internalization following 1 hour of incubation at 37° C. of 50 μl of liposome without CPP. Membrane and nuclei markers used are the same as those described in FIG. 12. Labeling with the word “red” in this image indicates the internalization of labeled liposomes inside the cell.

FIG. 16 presents a snapshot image of a stained subpopulation. FIGS. 16A and 16B demonstrate an originally light blue labeling of the mitochondria. FIGS. 16C and 16D indicate an originally red labeling of the Lysosomes. FIGS. 16E and 16F indicate an originally green labeling of S22 (SEQ ID NO 1) and S24 (SEQ ID NO 2) respectively. FIGS. 16G and 16H indicate an originally blue labeling of the nucleus.

FIG. 17 shows results of kinetic profiling of CPPs: S22 and S24 (SEQ ID NOs 1 and 2, respectively) on cells of the A549 cell line. Panels A1 and B1 indicate labeling agent intensity in cell population at time zero (background intensity) for S22 and S24 respectively. Labeling agent intensity demonstrates the amount of CPPs internalized cells. Panels A2 and B2 indicate intensity after 30 minutes incubation with labeled S22 and S24 CPPs respectively. Panels A3 and B3 indicates intensity after 45 minutes, a4 and B4 after 60 minutes and A5 and B5 after 90 minutes, all of S22 and S24 respectively.

### DETAILED DESCRIPTION OF THE INVENTION

Novel peptides were identified that were found to be suitable as cell-penetrating peptides (CPP), which may deliver cargo molecules inside the cell. CPP enables the delivery of such cargo molecules to the cytoplasm, nucleolus or any organelle inside the cell. CPP peptides, variants thereof and/or complexes, fusions and/or conjugates comprising same can be used for manufacture of pharmaceutical compositions for treating of a wide range of conditions, disorders and diseases. CPP peptides, variants thereof and/or complexes, fusions and/or conjugates comprising same can be further used for manufacture of diagnostic compositions for diagnosing of a wide range of conditions, disorders and diseases. The CPPs and/or variants and/or complexes, fusions, and/or conjugates and/or pharmaceutical compositions comprising same can be used for treating and/or preventing of a wide range of conditions, disorders and diseases. The CPPs and/or variants and/or complexes, fusions and/or conjugates and/or pharmaceutical compositions comprising same can be used for diagnosing of a wide range of conditions, disorders and diseases.

Below an explanation is provided for at least some embodiments, which indicate that variants may optionally be determined (in whole or in part) according to a sliding window relative to a base protein from which the CPP peptide sequence is derived; without wishing to be limited by a single hypothesis, different portions of the base protein may optionally provide different CPP functionality within the limits described below. It should be noted that the functionality of the resultant CPP peptide is not limited by the identity of the putative base protein.

In at least some embodiments, the subject invention provides a peptide consisting essentially of an amino acid sequence KKTCRRINYCALNK (S22 [SEQ ID NO: 1]) or a variant thereof. S22 corresponds to amino acid residues 397-410 of the MATN2_HUMAN protein sequence (SwissProt Accession number: O00339, SEQ ID NO: 13).

In at least some embodiments, the subject invention thus provides a peptide consisting essentially of an amino acid sequence KKTLKKLWKKKKRK (S24 [SEQ ID NO: 2]) or a variant thereof. S24 corresponds to amino acid residues 373-386 of the CAPSD_FMVD protein sequence (SwissProt Accession number: P09519, SEQ ID NO: 14).

In at least some embodiments, the subject invention thus provides a peptide consisting essentially of an amino acid sequence RRRHQRKYRRY (S36 [SEQ ID NO: 3]) or a variant thereof. S36 corresponds to amino acid residues 67-77 of the CA063_HUMAN protein sequence (SwisProt Accession number: Q9BUV0, SEQ ID NO: 15).

In at least some embodiments, the subject invention thus provides a peptide consisting essentially of an amino acid sequence FLRRRRRFTRQT (S40 [SEQ ID NO: 4]) or a variant thereof. S40 corresponds to amino acid residues 107-118 of the FOXS1_HUMAN protein sequence (SwisProt Accession number: O43638, SEQ ID NO: 16).

In at least some embodiments, the subject invention thus provides a peptide consisting essentially of an amino acid sequence KKLALYLLLAL (S67 [SEQ ID NO: 5]) or a variant thereof. S67 corresponds to amino acid residues 2425-2435 of the POLG_YEFVT protein sequence (SwisProt Accession number: Q9YRV3, SEQ ID NO: 17).

In at least some embodiments, the subject invention thus provides a peptide consisting essentially of an amino acid sequence KPSARMLLLKGFGK (S8 [SEQ ID NO: 6]) or a variant thereof. S8 corresponds to amino acid residues 91-104 of the PNPO_HUMAN protein sequence (SwisProt Accession number: Q9NVS9, SEQ ID NO: 18).

In order that the present invention may be more readily understood, certain terms and definitions are hereby provided:

As used herein, the term “CPP(s)” or “CPP peptide(s)” (used herein interchangeably) refers to a peptide according to at least some embodiments, having an amino acid sequence selected from any one of SEQ ID NOs: 1-6, variants thereof, complexes, fusions and/or conjugates thereof. Such a peptide preferably has the functional activity of a cell-penetrating peptide. As used herein, the term “CPP(s)” further refers to non-limiting examples of variant peptides having an amino acid sequence selected from any one of SEQ ID NOs: 7-12 and 33, or further variants thereof.

As used herein, the term “cargo” or “cargo molecule” refers to an entity which is transported or more efficiently delivered inside the cell using any one of the CPPs. The cargo molecule optionally includes, but is not limited to, one or more of any one of nucleic acids, oligonucleotides such as siRNA, dsRNA, miRNA, DNA, RNA, PNA, antisensemolecules, ribozymes, aptamers, spiegelmers, decoy molecules, antibodies, amino acids, peptides, proteins, lipids, carbohydrates, small molecules and combinations thereof. The various cargo types and alternatives are described in more detail in the “CARGO TYPES AND ALTERNATIVES” section herein.

The cargo molecule may optionally comprise any type of cellular effector. A cellular effector can herein be either an intracellular and/or extracellular effector and is in the present context defined as a molecule that produces a cellular effect, such as a contraction, secretion, electrical impulse, or activation or inactivation of an intracellular and/or extracellular signalling cascade, or that induces the up regulation of a cellular level of an mRNA and/or a protein, in response to a stimulation by said effector. A typical effector is in the present context selected from the group consisting of a metabolite, an antagonist, an agonist, a receptor ligand, a receptor coupled protein, an activated receptor, an enzyme inhibitor, activator/inactivator and/or stimulator, a kinase, a phosphatase, an enhancer, or a silencer, a transcription factor, a transporter and/or a transmitter, a hormone, a channel, an ion, a prion, and a viral protein.

Optionally, according to at least some embodiments of the invention, any one of the CPPs attached to a cargo is a cellular effector.

The term “Complex”, as used herein, refers to an entity including a CPP or CPPs and at least one type of other cargo molecule which are not covalently bonded (e.g. electrostatic interaction, hydrogen bonds, ionic bonds, and hydrophobic interactions). Such a complex may be produced by physical association (e.g. siRNA and a cationic CPP in a solution), encapsulation within a third entity (e.g. liposome) or any other methodology known in the art which produces a measurable to certain extent association between the CPP and the other cargo. Additional molecules may be associated to form such complexes (e.g. His amino acids). As used herein, the term “complex” refers also to CPP and/or cargo which is non-covalently attached to a fusion or a conjugate.

The term “Complexation” or “Association” as used herein, refers to the act of creating a complex.

The terms “Fusion” or “Conjugate” as used herein, refer to a CPP or CPPs that are covalently bond (e.g. a peptide bond, disulfide bond) to at least one type of cargo molecule.

The terms “Conjugation”, “covalently linking” or “covalently bonding” as used herein refer to the act of creating a fusion entity.

As used herein, the term “attachment” or “attaching” refers to conjugation, fusion, complexation or association between at least two molecular entities (e.g. CPP peptide and an siRNA) which may be referred to as a single entity.

As used herein, the term “linkage group” refers to any molecular entity, such as polymer, peptide, hydrophobic or polar peptide, protein, lipid, dendrimers, etc., which links two molecular entities preferably by a covalent bond.

As used herein, the term “prodrug” refers to an entity that needs to be modified in order to become active and preferably function as a therapeutic entity or as a functional delivery agent/entity that transports a cargo molecule into a cell. As used herein, the term “enzyme-prodrug” refers to a prodrug entity which becomes active or functional as a consequence of chemical modification by an enzyme, preferably enzymatic cleavage.

As used herein, the term “homing entity” or “homing peptide” refers to a molecule which has a preferred selectivity outcome based on binding affinity or cleavage at site of tissue or other activity for a cell, cell type, specific tissue, group of tissues, organ, location or a defined object. The terms “homing” and “targeting” are used herein interchangeably.

As used herein the term “polypeptide” refers to a molecule comprising at least 2 amino acids. The term “polypeptide” is to be understood to include, inter alia, native peptides (either degradation products, synthetically synthesized peptides or recombinant peptides), peptidomimetics, such as peptoids and semipeptoids or peptide analogs, which may comprise, for example, any desirable modification, including, inter alia, modifications rendering the peptides more stable while in a body or more capable of penetrating into cells, or others as will be appreciated by one skilled in the art. Such modifications include, but are not limited to chemical modification, N terminus modification, C terminus modification, peptide bond modification, backbone modifications, residue modification, D-amino acids, or non-natural amino acids or others.

As used herein the term “treatment” refers to care provided to relieve illness and refers to both a therapeutic treatment or prophylactic/preventative measures, wherein the objective is to prevent or slow down (lessen) the targeted pathologic condition or disorder. Those in need of treatment include those already with the disorder as well as those prone to have the disorder or those in whom the disorder is to be prevented. The term treatment as used herein refers also to “maintenance therapy”, which is a treatment that is given to keep a pathologic condition or disorder from coming back after it has disappeared following the initial therapy. As used herein the term “treating” further refers to preventing, curing, reversing, attenuating, alleviating, minimizing, suppressing or halting the deleterious effects of the diseases, disorders or conditions.

It will be appreciated that treatment according to at least some embodiments of the present invention may be combined with other treatment methods known in the art (i.e., combination therapy).

As used herein the term “diagnosis” refers to the process of identifying a medical condition or disease by its signs, symptoms, and in particular from the results of various diagnostic procedures, including e.g. detecting the expression of the nucleic acids or polypeptides according to at least some embodiments of the invention in a biological sample (e.g. in cells, tissue or serum, as defined below) obtained from an individual. Furthermore, as used herein the term “diagnosis” encompasses screening for a disease, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. The diagnostic procedure can be performed in vivo or in vitro.

As used herein the term “combination therapy” refers to the simultaneous or consecutive administration of two or more medications or types of therapy to treat a single disease. In particular, the term refers to the use of any of the polypeptides, polynucleotides, antibodies or pharmaceutical compositions according to at least some embodiments of the invention in combination with at least one additional medication or therapy. Thus, treatment of a disease using the agents according to at least some embodiments of the present invention may be combined with therapies well known in the art that include, but are not limited to, radiation therapy, antibody therapy, chemotherapy or surgery or in combination therapy with other biological agents, conventional drugs, anti-cancer agents, immunosuppressants, cytotoxic drugs for cancer, chemotherapeutic agents.

As used herein, the term “subject” or “patient” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The term “fragment” relating to the peptides of at least some embodiments of the invention as used herein should be understood to encompass peptides which have cell penetrating biological activity as for the CPPs set forth in any one of SEQ ID NOs: 1-6, yet which have different sequences in that these sequences represent a consecutive portion of the amino acid sequence of any of SEQ ID NOs: 1-6.

The term “homolog” relating to the peptides of at least some embodiments of the invention as used herein should be understood to encompass peptides which have cell penetrating biological activity as for the CPPs set forth in any one of SEQ ID NOs: 1-6, yet which have different sequences. Thus, a homolog may differ from the sequence of the CPPs set forth in any one of SEQ ID NOs: 1-6 by the addition, deletion or substitution of one or more amino acid residues, provided that the resulting peptide retains the cell penetrating activity of the CPPs. Persons skilled in the art can readily determine which amino acid residues may be added, deleted or substituted (including with which amino acids such substitutions may be made) using established well known procedures. Examples of homologs of the CPPs are deletion variants containing less than all the amino acid residues of any one of SEQ ID NOs: 1-6 (in which the deletion results in a peptide having a non-consecutive portion of the amino acid sequence of any of SEQ ID NOs: 1-6), substitution variants wherein one or more amino acid residues specified are replaced by other amino acid residues (eg. amino acid with similar properties or by D-amino acids, or by non-natural amino acids) and addition variants wherein one or more amino acid residues are added to a terminal or medial portion of any one of SEQ ID NOs:1-6, all of which share the biological activity of any one of SEQ ID NOs: 1-6. The term “homolog” relating to a peptide of the invention as used herein should be understood to encompass an ortholog. The term “ortholog” should be understood to encompass a peptide derived from a other origin than the shown origin (eg. non-human or non-viral origin) which has substantially the same amino acid sequence and substantially the same biological activity as CPPs set forth in any one of SEQ ID NOs:1-6.

In one embodiment, a variant of S22 peptide according to at least some embodiments of the present invention is CLKGFTLNPDKKTCRRINYCALNKPGCEHECVNM [SEQ ID NO: 7] which corresponds to amino acid residues 387-420 of the MATN2_HUMAN protein sequence (SwissProt Accession number: O00339, SEQ ID NO: 13), or a derivative thereof.

In one embodiment, a variant of S24 peptide according to at least some embodiments of the present invention is KSSKYKAYKKKKTLKKLWKKKKRKFTPGKYFSKK [SEQ ID NO: 8], which corresponds to amino acid residues 363-396 of the CAPSD_FMVD protein sequence (SwissProt Accession number: P09519, SEQ ID NO: 14), or a derivative thereof.

In one embodiment, a variant of S24 peptide according to at least some embodiments of the present invention is PLVSFGCRDTKKKDFKKSSKYKAYKKKKTLKKLWKKKKRKFTPGKYFSKK (SEQ ID NO:33), which corresponds to amino acid residues 347-396 of the CAPSD_FMVD protein sequence (SwissProt Accession number: P09519, SEQ ID NO: 14), or a derivative thereof.

In one embodiment, a variant of S36 peptide according to at least some embodiments of the present invention is SRRSKSRSRSRRRHQRKYRRYSRSYSRSRSR [SEQ ID NO: 9], which corresponds to amino acid residues 57-87 of the CA063_HUMAN protein sequence (SwisProt Accession number: Q9BUV0, SEQ ID NO: 15), or a derivative thereof.

In one embodiment, a variant of S40 peptide according to at least some embodiments of the present invention is DCHDMFEHGSFLRRRRRFTRQTGAEGTRGPAK [SEQ ID NO: 10], which corresponds to amino acid residues 97-128 of the FOXS1_HUMAN protein sequence (SwisProt Accession number: O43638, SEQ ID NO: 16), or a derivative thereof.

In one embodiment, a variant of S67 peptide according to at least some embodiments of the present invention is EAPEMPALYEKKLALYLLLALSLASVAMCRT [SEQ ID NO: 11], which corresponds to amino acid residues 2415-2445 of the POLG_YEFVT protein sequence (SwisProt Accession number: Q9YRV3, SEQ ID NO: 17), or a derivative thereof.

In one embodiment, a variant of S8 peptide according to at least some embodiments of the present invention is MCLATCTRDGKPSARMLLLKGFGKDGFRFFTNFE [SEQ ID NO: 12], which corresponds to amino acid residues 81-114 of the PNPO_HUMAN protein sequence (SwisProt Accession number: Q9NVS9, SEQ ID NO: 18), or a derivative thereof.

The term “derivative” relating to a peptide of the invention should be understood to encompass a peptide which has substantially the same amino acid sequence as CPPs set forth in any one of SEQ ID NOs:1-6 but with some type of chemical modification. Thus, a derivative may differ from any one of the SEQ ID NOs: 1-6 peptides by a modification, such as but not limited to glycosylation, amidation, acetylation, alkylation, alkenylation, alkynylation, phosphorylation, sulphorization, hydroxylation, hydrogenation, cyclization and so forth. Thus, a derivative of a peptide of the invention may differ from any one of the SEQ ID NOs:1-6 peptide (or indeed optionally any peptide described herein according to various embodiments of the present invention) by a modification of one or more amino acid residues, provided that the resulting peptide retains the cell penetrating activity of CPPs set forth in any one of SEQ ID NOs: 1-6. Persons skilled in the art can readily determine which amino acid residues may be modified using established well known procedures. In one embodiment, a peptide of the invention is amidated at its C-terminus and acetylated at its N-terminus.

Non-limiting examples of other variant peptides according to various embodiments of the present invention are given below.

“A peptide with substantially the same amino acid sequence as SEQ ID NO:1” as used herein should be understood to encompass a synthetic peptide which has at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, and at most 34 amino acids, which correspond to a sequential fragment of amino acid residues 387-420 of the MATN2_HUMAN protein sequence (SwissProt Accession number: O00339, SEQ ID NO: 13). More preferably “A peptide with substantially the same amino acid sequence as SEQ ID NO:1” as used herein should be understood to encompass a synthetic peptide which has at least 14 amino acids which correspond to a sequential fragment of amino acid residues 397-410 of the MATN2_HUMAN protein sequence (SwissProt Accession number: O00339, SEQ ID NO: 13).

“A peptide with substantially the same amino acid sequence as SEQ ID NO:2” as used herein should be understood to encompass a synthetic peptide which has at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, and at most 50 amino acids, which correspond to a sequential fragment of amino acid residues 346-396 of the CAPSD_FMVD protein sequence (SwissProt Accession number: P09519, SEQ ID NO: 14). More preferably “A peptide with substantially the same amino acid sequence as SEQ ID NO:2” as used herein should be understood to encompass a synthetic peptide which has at least 14 amino acids which correspond to a sequential fragment of amino acid residues 373-386 of the CAPSD_FMVD protein sequence (SwissProt Accession number: P09519, SEQ ID NO: 14).

“A peptide with substantially the same amino acid sequence as SEQ ID NO:3” as used herein should be understood to encompass a synthetic peptide which has at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, and at most 31 amino acids, which correspond to a sequential fragment of amino acid residues 57-87 of the CA063_HUMAN protein sequence (SwisProt Accession number: Q9BUV0, SEQ ID NO: 15). More preferably “A peptide with substantially the same amino acid sequence as SEQ ID NO:3” as used herein should be understood to encompass a synthetic peptide which has at least 11 amino acids which correspond to a sequential fragment of amino acid residues 67-77 of the CA063_HUMAN protein sequence (SwisProt Accession number: Q9BUV0, SEQ ID NO: 15).

“A peptide with substantially the same amino acid sequence as SEQ ID NO:4” as used herein should be understood to encompass a synthetic peptide which has at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, and at most 32 amino acids, which correspond to a sequential fragment of amino acid residues 97-128 of the FOXS1_HUMAN protein sequence (SwisProt Accession number: O43638, SEQ ID NO: 16). More preferably “A peptide with substantially the same amino acid sequence as SEQ ID NO:4” as used herein should be understood to encompass a synthetic peptide which has at least 12 amino acids which correspond to a sequential fragment of amino acid residues 107-118 of the FOXS1_HUMAN protein sequence (SwisProt Accession number: O43638, SEQ ID NO: 16).

“A peptide with substantially the same amino acid sequence as SEQ ID NO:5” as used herein should be understood to encompass a synthetic peptide which has at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, and at most 31 amino acids, which correspond to a sequential fragment of amino acid residues 2415-2445 of the POLG_YEFVT protein sequence (SwisProt Accession number: Q9YRV3, SEQ ID NO: 17). More preferably “A peptide with substantially the same amino acid sequence as SEQ ID NO:5” as used herein should be understood to encompass a synthetic peptide which has at least 11 amino acids which correspond to a sequential fragment of amino acid residues 2425-2435 of the POLG_YEFVT protein sequence (SwisProt Accession number: Q9YRV3, SEQ ID NO: 17).

“A peptide with substantially the same amino acid sequence as SEQ ID NO:6” as used herein should be understood to encompass a synthetic peptide which has at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, and at most 34 amino acids, which correspond to a sequential fragment of amino acid residues 81-114 of the PNPO_HUMAN protein sequence (SwisProt Accession number: Q9NVS9, SEQ ID NO: 18). More preferably “A peptide with substantially the same amino acid sequence as SEQ ID NO:6” as used herein should be understood to encompass a synthetic peptide which has at least 14 amino acids which correspond to a sequential fragment of amino acid residues 91-104 of the PNPO_HUMAN protein sequence (SwisProt Accession number: Q9NVS9, SEQ ID NO: 18).

“A peptide with substantially the same biological activity as CPP set forth in any one of SEQ ID NOs: 1-6” as used herein should be understood to encompass a peptide which has at least 80% of the biological activity of CPPs set forth in any one of SEQ ID NOs 1-6, respectively. A “biological activity” as used herein should be understood to encompass a cell penetrating activity of the peptide such those described in this document of fluorescently labeled CPPs internalization measured by flow cytometry or confocal microscopy (e.g. the assay presented in Example 2 of flow cytometry analysis produces the geometric mean FL1 intensity of each examined cell population on HeLa cells with each and every CPP from SEQ ID NOs 1-6. A reporter agent intensity that is 80% of such a geometric mean resembles a situation where the geometric mean of a population of cells with another peptide produces at least factor of 0.8 of the geometric mean of the equivalent CPP). More preferably intracellular cargo delivery of the peptide such pro-apoptotic peptide as cargo, attached to the CPPs where the penetration activity is measured by the cell viability as described in this document in the example section, inter alia, tissue specific or targeting capability.

A “diagnostic marker” as used herein should be understood to encompass any entity which comprises at least a labeling and a targeting entity or entities with a special characteristic of selectivity to a certain pathological cell(s), tissue or tissues over corresponding normal cell(s), tissue or tissues, preferably a cancerous cell(s), tissue or tissues over non cancerous normal cell(s), tissue or tissues, preferably in human body. A diagnostic marker for example can be introduced to a patient to visualize a bio distribution of the pathological cell(s), tissue or tissues in the patient's body. The visualization of the pathological cell(s), tissue or tissues in the patient's body is carried out using any one of the in vivo imaging techniques known in the art.

Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

A peptide according to at least some embodiments of the invention may be prepared synthetically (e.g. on a solid support by solid phase peptide synthesis or in solution) or by recombinant means (in bacteria, yeast, fungi, insect, vertebrate or mammalian cells) by methods well known to those skilled in the art.

It will be appreciated that peptides identified according to the teachings at least some embodiments of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

In one embodiment, a peptide of the invention may be synthesized such that one or more of the bonds which link the amino acid residues of the peptide, are non-peptide bonds.

In another embodiment, a peptide of the invention may be synthesized with additional chemical groups, such that, for example, the stability, bioavailability, membrane penetration ability, cargo delivery, specific tissue activity and/or inhibitory activity of the peptide is modified. For example, an acetyl group may be placed at the amino termini of a peptide according to at least some embodiments of the invention. Additionally or alternatively, an amido group may be added to the carboxy termini of a peptide according to at least some embodiments of the invention. Additionally or alternatively, a Folate molecule or beta-glucan may be added to the peptide.

Natural aromatic amino acids, Trp, Tyr and Phe, may be substituted by synthetic non-natural acid such as Phenylglycine, TIC, naphthylelanine (Nol), ring-methylated derivatives of Phe, halogenated derivatives of Phe or o-methyl-Tyr.

In addition to the above, the peptides according to at least some embodiments of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

In yet a further embodiment, at least one of the amino acid residues of a peptide according to at least some embodiments of the invention may be substituted by any one of the well known non-naturally occurring amino acid residues.

Non-natural amino acids are known to those skilled in the art of chemical synthesis and peptide chemistry. Non-limiting examples of non-natural amino acids (each one in L- or D-configuration) are azidoalanine, azidohomoalanine, 2-amino-5-hexynoic acid, norleucine, azidonorleucine, L-a-aminobutyric acid, 3-(1-naphthyl)-alanine, 3-(2-naphthyl)-alanine, p-ethynyl-phenylalanine, m-ethynyl-phenylalanine, p-ethynyl-phenylalanine, p-bromophenylalanine, p-idiophenylalanine, p-azidophenylalanine, 3-(6-chloroindolyl) alanin and those listed in Table 1 below.

In yet a further embodiment, at least one of the amino acid residues of a CPP according to at least some embodiments of the invention may be substituted by any one of the well known conservative substitutions, such as these that appear for example in Table 2 herein. Amino acids in the same block in the second column of Table 2 may be substituted for each other.

TABLE 1

| Non-conventional amino | | Non-conventional amino | |
|---|---|---|---|
| acid | Code | acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3- | Nbhe |
| | | diphenylpropyl)glycine | |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N- | Nmchexa | D-N-methylmethionine | Dnmmet |
| methylcyclohexylalanine | | | |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3- | Narg |
| | | guanidinopropyl)glycine | |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N- | Nmchexa | D-N-methylmethionine | Dnmmet |
| methylcyclohexylalanine | | | |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α- | Mhphe |
| | | methylhomophenylalanine | |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N- | Nmhphe |
| | Nnbhm | methylhomophenylalanine | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

TABLE 2
Conservative substitutions

| Conservative Group | Amino Acids |
|---|---|
| ALIPHATIC Non-polar | G A P I L V |
| Polar - uncharged | C S T M N Q |
| Polar - charged + | K R |
| Polar - charged − | D E |
| AROMATIC | H F W Y |

Since the peptides according to at least some embodiments of the present invention are preferably utilized in therapeutics which require the peptides to be in soluble form, the peptides according to at least some embodiments of the present invention preferably include one or more non-natural or natural polar amino acids, including but not limited to serine and threonine which are capable of increasing peptide solubility due to their hydroxyl-containing side chain.

The peptides according to at least some embodiments of the present invention are preferably utilized in a linear form, although it will be appreciated that in cases where cyclization does not severely interfere with peptide characteristics, cyclic forms of the peptide can also be utilized.

In another embodiment, a peptide according to at least some embodiments of the invention may have a non-peptide macromolecular group covalently attached to its amino and/or carboxy terminus. Non-limiting examples of such macromolecular groups are proteins, lipid-fatty acid, polyethylene glycol, and carbohydrates.

A “peptidomimetic organic moiety” can optionally be substituted for amino acid residues in the composition according to at least some embodiments of this invention both as conservative and as non-conservative substitutions. These moieties are also termed “non-natural amino acids” and may optionally replace amino acid residues, amino acids or act as spacer groups within the peptides in lieu of deleted amino acids. The peptidomimetic organic moieties optionally and preferably have steric, electronic or configurational properties similar to the replaced amino acid and such peptidomimetics are used to replace amino acids in the essential positions, and are considered conservative substitutions. However such similarities are not necessarily required. According to at least some embodiments of the present invention, one or more peptidomimetics are selected such that the composition at least substantially retains its physiological activity as compared to the native peptide according to at least some embodiments of the present invention.

Peptidomimetics may optionally be used to inhibit degradation of the peptides by enzymatic or other degradative processes. The peptidomimetics can optionally and preferably be produced by organic synthetic techniques. Non-limiting examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable but exemplary peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable exemplary peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S,3S)-methyl-phenylalanine, (2S,3R)-methyl-phenylalanine, (2R,3S)-methyl-phenylalanine and (2R,3R)-methyl-phenylalanine (Kazmierski and Hruby, Tetrahedron Lett. (1991)).

Exemplary, illustrative but non-limiting non-natural amino acids include beta-amino acids (beta3 and beta2), homo-amino acids, cyclic amino acids, aromatic amino acids, Pro and Pyr derivatives, 3-substituted Alanine derivatives, Glycine derivatives, ring-substituted Phe and Tyr Derivatives, linear core amino acids or diamino acids. They are available from a variety of suppliers, such as Sigma-Aldrich (USA) for example.

All amino acid sequences and nucleic acid sequences shown herein as embodiments of the present invention relate to their isolated form.

Targeting Fusion Peptides

According to at least some embodiments of the present invention, a fusion peptide may be prepared from a peptide according to at least some embodiments of the invention by fusion with a portion of an immunoglobulin comprising a constant region of an immunoglobulin. Such an immunoglobulin is relevant for extracellular and intracellular targeting, and also optionally for therapeutic purposes. More preferably, the portion of the immunoglobulin comprises a heavy chain constant region which is optionally and more preferably a human heavy chain constant region. The heavy chain constant region is most preferably an IgG heavy chain constant region, and optionally and most preferably is an Fc chain, most preferably an IgG Fc fragment that comprises CH2 and CH3 domains. Although any IgG subtype may optionally be used, the IgG1 subtype is preferred. The Fc chain may optionally be a known or “wild type” Fc chain, or alternatively may be mutated. Non-limiting, illustrative, exemplary types of mutations are described in US Patent Application No. 20060034852, published on Feb. 16, 2006, hereby incorporated by reference as if fully set forth herein. The term “Fc chain” also optionally comprises any type of Fc fragment.

Several of the specific amino acid residues that are important for antibody constant region-mediated activity in the IgG subclass have been identified. Inclusion, substitution or exclusion of these specific amino acids therefore allows for inclusion or exclusion of specific immunoglobulin constant region-mediated activity. Furthermore, specific changes may result in aglycosylation for example and/or other desired changes to the Fc chain. At least some changes may optionally be made to block a function of Fc which is considered to be undesirable, such as an undesirable immune system effect, as described in greater detail below.

Non-limiting, illustrative examples of mutations to Fc which may be made to modulate the activity of the fusion peptide include the following changes (given with regard to the Fc sequence nomenclature as given by Kabat, from Kabat E A et al: Sequences of Peptides of Immunological Interest. US Department of Health and Human Services, NIH, 1991): 220C->S; 233-238 ELLGGP->EAEGAP; 265D->A, preferably in combination with 434N->A; 297N->A (for example to block N-glycosylation); 318-322 EYKCK->AYACA; 330-331AP->SS; or a combination thereof (see for example M. Clark, “Chemical Immunol and Antibody Engineering”, pp 1-31 for a description of these mutations and their effect). The construct for the Fc chain which features the above changes optionally and preferably comprises a combination of the hinge region with the CH2 and CH3 domains.

The above mutations may optionally be implemented to enhance desired properties or alternatively to block non-desired properties. For example, aglycosylation of antibodies was shown to maintain the desired binding functionality while blocking depletion of T-cells or triggering cytokine release, which may optionally be undesired functions (see M. Clark, “Chemical Immunol and Antibody Engineering”, pp 1-31). Substitution of 331 proline for serine may block the ability to activate complement, which may optionally be considered an undesired function (see M. Clark, “Chemical Immunol and Antibody Engineering”, pp 1-31). Changing 330alanine to serine in combination with this change may also enhance the desired effect of blocking the ability to activate complement.

Residues 235 and 237 were shown to be involved in antibody-dependent cell-mediated cytotoxicity (ADCC), such that changing the block of residues from 233-238 as described may also block such activity if ADCC is considered to be an undesirable function.

Residue 220 is normally a cysteine for Fc from IgG1, which is the site at which the heavy chain forms a covalent linkage with the light chain. Optionally, this residue may be changed to a serine, to avoid any type of covalent linkage (see M. Clark, “Chemical Immunol and Antibody Engineering”, pp 1-31).

The above changes to residues 265 and 434 may optionally be implemented to reduce or block binding to the Fc receptor, which may optionally block undesired functionality of Fc related to its immune system functions (see “Binding site on Human IgG1 for Fc Receptors”, Shields et al, Vol 276, pp 6591-6604, 2001).

The above changes are intended as illustrations only of optional changes and are not meant to be limiting in any way. Furthermore, the above explanation is provided for descriptive purposes only, without wishing to be bound by a single hypothesis.

Non-Limiting Examples of Derivatives

If a peptide according to at least some embodiments of the present invention is a linear molecule, it is possible to place various functional groups at various points on the linear molecule which are susceptible to or suitable for chemical modification. Functional groups can be added to the termini of linear forms of the peptide according to at least some embodiments of the invention. In some embodiments, the functional groups improve the activity of the peptide with regard to one or more characteristics, including but not limited to, improvement in stability, penetration (through cellular membranes and/or tissue barriers), tissue localization, efficacy, decreased clearance, decreased toxicity, improved selectivity, improved resistance to expulsion by cellular pumps, and the like. For convenience sake and without wishing to be limiting, the free N-terminus of one of the sequences contained in the compositions according to at least some embodiments of the invention will be termed as the N-terminus of the composition, and the free C-terminal of the sequence will be considered as the C-terminus of the composition. Either the C-terminus or the N-terminus of the sequences, or both, can be linked to a carboxylic acid functional groups or an amine functional group, respectively.

Non-limiting examples of suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moiety for transport across cellular membranes”.

These moieties can optionally and preferably be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a composition according to at least some embodiments of the present invention is protected, preferably with a methyl, ethyl, benzyl or substituted benzyl ester, more preferably as a benzyl ester.

Non-limiting, illustrative examples of N-terminal protecting groups include acyl groups (—CO—R1) and alkoxy carbonyl or aryloxy carbonyl groups (—SO—O—R1), wherein R1 is an aliphatic, substituted aliphatic, benzyl, substituted benzyl, aromatic or a substituted aromatic group. Specific examples of acyl groups include but are not limited to acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—OO—, Adamantan, naphtalen, myristoleyl, toluen, biphenyl, cinnamoyl, nitrobenzoy, toluoyl, furoyl, benzoyl, cyclohexane, norbornane, or Z-caproic. In order to facilitate the N-acylation, one to four glycine residues can be present in the N-terminus of the molecule.

The carboxyl group at the C-terminus of the compound can be protected, for example, by the group including but not limited to an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are optionally independently an aliphatic, substituted aliphatic, benzyl, substituted benzyl, aryl or a substituted aryl group. In addition, taken together with the nitrogen atom, R₂ and R₃ can optionally form a C4 to C8 heterocyclic ring with from about 0-2 additional heteroatoms such as nitrogen, oxygen or sulfur. Non-limiting suitable examples of suitable heterocyclic rings include piperidinyl, pyrrolidinyl, morpholino, thiomorpholino or piperazinyl. Examples of C-terminal protecting groups include but are not limited to —NH₂, —NHCH₃, —N(CH₃)₂, —NH(ethyl), —N(ethyl)₂, —N(methyl) (ethyl), —NH(benzyl), —N(C1-C4 alkyl) (benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

In the present invention, according to at least some embodiments thereof, any part of a peptide of the invention may optionally be chemically modified, i.e. changed by addition of functional groups. Optionally, the peptide for such embodiments may be considered as a combination of a peptidic core plus one or more modifications. For example the side amino acid residues appearing in the native sequence may optionally be modified, although as described below alternatively other parts of the peptide may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible.

The amino acid of any of the sequence regions of the molecule can optionally be modified according to any one of the following exemplary types of modification (in the peptide conceptually viewed as “chemically modified”). For example optionally any type of suitable moiety or molecule may be covalently bound to the amino acid and/or otherwise bound to the peptide. Non-limiting examples of such groups include cysteamide, a cysteine, a thiol, an amide, a carboxyl, a linear or ramified C1-C6 alkyl optionally substituted, a primary or secondary amine, an osidic derivative, a lipid, a phospholipid, a fatty acid, a cholesterol, a poly-ethylene glycol, a nuclear localization signal (NLS), and/or a targeting molecule. Alternatively or additionally, the cell-penetrating peptide of the invention may also optionally comprise, for example covalently linked to the N-terminal end of the peptidic core of the CPP, one or more chemical entities selected from the group consisting of an acetyl, a fatty acid, a cholesterol, a poly-ethylene glycol, a nuclear localization signal, and/or a targeting molecule. If necessary, for example in the case of N-terminal addition of cholesterol, a peptidic bridge may optionally be used to bind a non-peptidic molecule to the peptidic core of the CPP.

Non-limiting exemplary types of modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, according to at least some embodiments of the present invention, refers to a peptide where at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a peptide, as described in PCT Application No. WO 2006/050262, hereby incorporated by reference as if fully set forth herein. These moieties are designed for use with biomolecules and may optionally be used to impart various properties to peptides.

Furthermore, optionally any point on a peptide may be modified. For example, pegylation of a glycosylation moiety on a peptide may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a peptide through a glycosyl linker.

Altered Glycosylation

Peptides according to at least some embodiments of the present invention, may optionally be modified to be glycosylated (if no native glycosylation occurs) and/or to have an altered glycosylation pattern (i.e., altered from the original or native glycosylation pattern). As used herein, “altered” means having one or more carbohydrate moieties deleted, and/or having at least one glycosylation site added to the original peptide. Glycosylation of bioactive compounds has been used mainly for increasing either the hydrophilicity, the enzymatic stability of the compound, and/or its delivery into the brain. In the context of CPPs, glycosylation may regulate and or affect the internalization of the corresponding glycosylated analogs. E.g. in [Dutot., et al., J Chem Biol. 2010 May; 3(2): 51-65], the effect on cell viability and the uptake efficiency of different glycosylated CPP complexes, fusions and/or conjugates were studied and compared to those of the nonglycosylated CPPs. Results suggested that glycosylation of CPP may be a key point in targeting specific cells.

Glycosylation of peptides is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to peptides according to at least some embodiments of the invention is conveniently accomplished by altering the amino acid sequence of the peptide such that it contains one or more of the above-described tripeptide sequences (for N-linked glycosylation sites). The alteration may also be made by the addition of, or substitution by, one or more serine or threonine residues in the sequence of the original peptide (for O-linked glycosylation sites). The peptide's amino acid sequence may also be altered by introducing changes at the DNA level.

Another means of increasing the number of carbohydrate moieties on peptides is by chemical or enzymatic coupling of glycosides to the amino acid residues of the peptide. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, CRC Crit. Rev. Biochem., 22: 259-306 (1981).

Removal of any carbohydrate moieties present on peptides according to at least some embodiments of the invention may be accomplished chemically or enzymatically. Chemical deglycosylation requires exposure of the peptide to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on peptides can be achieved by the use of a variety of endo- and exo-glycosidases as described by Thotakura et al., Meth. Enzymol., 138: 350 (1987).

### Internalization Characterization

The activity of the peptide according to at least some embodiments of the invention as a CPP, or CPP activity, may be determined by conjugation of the respective peptide to a fluorophore such as FAM (Carboxyfluorescein) that in this case serves both as a reporter group and as a cargo molecule, and enables the detection and quantification of cellular uptake by methods known to those familiar with the art. Cellular uptake may optionally be determined by quantifying the amount of CPP with cargo, without cargo or only cargo inside the cell, within certain compartments as nucleus, cytoplasm or any other. Such a method should distinguish between intracellular and extracellular compartmentalization (e.g. confusing a non specific binding of CPP to extracellular membrane as intracellular localization using a subjective detection method will lead to misleading uptake conclusions) and distinguishing between CPP intracellular delivery capability, cargo ability to penetrate the cell and synergistic effect (e.g. in the example described below, a pro apoptotic peptide is delivered into the cell. Intracellular uptake of that peptide using CPPs is assessed by cell viability assay while the pro-apoptotic peptide by itself does not penetrate cellular membrane, thus cells remain viable). Such methods include but are not limited to (i) flow cytometry and fluorescent microscopy for fluorophores serving as reporter group or (ii) fixation and permeabilization of cells followed by incubation with a reagent suitable for the detection by a functional assay as for the previously described cell viability assay, RNA silencing or any other suitable assay. Alternatively, the CPP may be radioactively labeled, e.g. by incorporation of radioactively labeled amino acids and the cellular uptake determined by radiography. The latter method enables the determination of uptake and distribution for the peptides alone, without any cargo. It is understood that to the degree the respective method so permits, the uptake and distribution may also be determined and quantitated for tissues and whole organisms. Alternatively, the uptake may be determined indirectly by means of the biological activity of a cargo molecule attached to the CPP and with the cargo molecule exerting its biological activity only if the molecule enters the cell and reaches a particular subcellular localization such as the cytoplasm or nucleus.

In a further embodiment, the peptides according to at least some embodiments of the present invention optionally comprise a moiety which is suitable for detection. More specifically, such moiety allows for the detection of the peptide. The moiety may be any group suitable for such purpose. Respective moieties are known to the ones skilled in the art and comprise, however, are not limited to, fluorophores, such as for example carboxyfluorescein, or biotin. Preferably the detection occurs by means of fluorescence. Alternatively, the detection may also occur by means of radioactivity, e.g. after incorporation of Iodine by protocols known to those skilled in the art. Detection may occur at the level of an individual cell, a tissue, an organ or an animal. Preferably the animal is a mammal and more preferably selected from the group comprising a dog, a cat, a sheep, a goat, a rat, a mouse, a cow, a horse and a human being.

### Cargo Types and Alternatives

In one embodiment the cargo molecule is a nucleic acid, wherein the nucleic acid is any polymer consisting of at least two nucleotides which are covalently linked. In one embodiment a nucleic acid can be a DNA molecule or a RNA molecule or a mixture thereof. It is also within at least some embodiments of the present invention that the nucleic acid consists of L-nucleotides, D-nucleotides or mixtures. In a further embodiment, the base moiety, the sugar moiety and/or the phosphate moiety of the individual nucleotide can be individually and independently modified for each and any of the nucleotides forming the nucleic acid or the respective analog. Particularly, preferred modified sugar moieties are those having a methyl, methoxy, ethyl or ethoxy group at the 2′ atom of the sugar moiety. Particularly, preferred modified phosphat moieties are phosphothioates. In another embodiment, peptide nucleic acids are employed.

In another embodiment the cargo molecule is an amino acid, originating from the group of L- or D-amino acids. The amino acid may be any amino acid, whether naturally occurring or non-natural.

In another preferred embodiment the cargo molecule is a peptide, consisting of at least two amino acids which are covalently linked, preferably through a peptide bond. In an embodiment the peptide consists of L-amino acids, D-amino acids or mixtures thereof. The amino acids may be any amino acids, whether naturally occurring or non-natural. In a preferred embodiment the term peptide thus also comprises peptides and proteins as generally understood in the art. The peptides or proteins may be purified from natural sources, obtained through organic synthesis or obtained by conjugation of synthetic amino acids or peptides to peptides or proteins obtained from natural sources by protocols familiar to those skilled in the art and exemplified but not limited to native chemical ligation. Preferably, peptides will have a length of 2 to 40 amino acids, more preferably of 2 to 30 amino acids, more preferably of 4 to 25, more preferably of 4 to 20, and more preferably of 4 to 17 amino acids. As used herein, the term protein preferably refers to a polypeptide containing secondary structure and more preferably tertiary structure.

In another embodiment the cargo molecule is a small molecule, whereby a small molecule is preferably a molecule having a molecular weight of 1000 Daltons or less and more preferably representing a drug or a drug candidate. Particularly preferred classes of small molecules are heterocyclic small molecules.

In another embodiment the cargo molecule is a lipid or a substructure of a lipid such as a moiety thereof. Preferably, a molecule of this class will exert a particular function once acting on the cell either inside or in the plasma membrane. An example for the former is diacylglycerol. This example illustrates that a lipid exerting such particular function is preferably selected from the group comprising intracellular messengers. An example for the latter and thus representing a possible cargo molecule is a lipopeptide, preferably a lipopeptide with a S-[2,3-bis(palmitoyloxy)-(2RS)-propyl]-N-palmitoyl-(R)-cysteinyl-(S)-seryl-tetra-(S)-lysine moiety and most preferably a peptide with a S-[2,3-bis(palmitoyloxy)-(2RS)-propyl]-N-palmitoyl-(R)-cysteinyl-(S)-seryl-tetra-(S)-lysine acting as an agonist or antagonist of a Toll-like receptor.

In another embodiment the cargo molecule is a carbohydrate.

In another embodiment the cargo is a contrasting agent used for magnetic resonance imaging. Such contrasting agents are for example but not limited to gadolinium (III)-DTPA (diethylenetriamine-pentaacetic acid) or gadolinium (III)-DOTA (1,4,7,10-Tetraazacyclododecane-1,4,7,10-tetraacetic acid)

In another embodiment, the cargo molecule is a particle. A particle may be a polymer particle, consisting, for example, of cross-linked polystyrene, cross-linked N-(2-hydroxypropyl)methacrylamide, cross-linked dextran, a liposome, or a micelle. Preferably, the particle serves as a carrier or container for a functional molecule. The functional molecule may be any molecules exerting a function inside cells, e.g. chemotherapeutics and oligonucleotides and preferably those that may also serve as cargo molecules for the CPPs according to at least some embodiments of the present invention. In general coupling of the functional molecule to the particle, respectively loading of the functional molecules into the particle, is intended to improve the pharmacokinetic properties of the functional molecule, e.g. by prolonging its circulation in the organism while coupling of the peptide(s) according to at least some embodiments of the present invention mediates the delivery of these functional molecules into cells. In addition to the peptide(s) according to at least some embodiments of the present invention, the particles may further be modified by a moiety or a molecule that mediate a targeting of the particles to specific cells. One example for such targeting are antibodies directed against proteins enriched on the surface of cancer cells. In one embodiment the particle may have a ferromagnetic core. Such particles may be used in applications such as magnetic fluid hyperthermia (Jordan et al., Int J Hyperthermia, 12, 705-722, 1996).

In another embodiment the cargo molecule is a quantum dot. Coupling of the CPPs according to at least some embodiments of the present invention to the quantum dot may be achieved by covalent coupling, for example by amide bond formation between suitable functionalities on the peptide and the quantum dot or by non covalent interactions, for example between a biotin moiety and a streptavidin molecule coupled to the quantum dot. In one example a cell-penetrating peptide is covalently linked to a quantum dot by elongation of the cell-penetrating peptide with a cysteine residue and coupling to amino-functionalized quantum dots using a heterobifunctional linker (S. Santra et al., Chem Comm, 2005, 3144-3146).

In a further embodiment, the cargo molecules can be defined in functional terms.

In a particular embodiment the cargo molecule is a siRNA molecule. siRNA molecules are small interfering RNAs directed to a target nucleic acid, preferably mRNA, coding for the target molecule. siRNA is a double stranded RNA having typically a length of about 21 to about 23 nucleotides. The sequence of one of the two RNA strands corresponds to the sequence of the target nucleic acid to be degraded. In other words, knowing the nucleic acid sequence of the target molecule, preferably the mRNA sequence, a double stranded RNA may be designed with one of the two strands being complementary to said mRNA of the target molecule and, upon application of said siRNA to a system containing the gene, genomic DNA, hnRNA or mRNA coding for the target molecule, the respective corresponding target nucleic acid will be degraded and thus the level of the respective protein be reduced. The basic principles of designing, constructing and using said siRNA as medicament and diagnostic agent, respectively, is, among others, described in international patent applications WO 00/44895 and WO 01/75164.

In a further embodiment the cargo molecule is a miRNA. miRNAs (MicroRNAs) are small, non-coding RNA molecules that occur naturally in human cells. They are post-transcriptional regulators that bind to complementary sequences in the three prime untranslated regions (3′ UTRs) of target messenger RNA transcripts (mRNAs), usually resulting in gene silencing. miRNAs are short ribonucleic acid (RNA) molecules, on average only 22 nucleotides long.

In a further embodiment the cargo molecule is a ribozyme. Ribozymes are catalytically active nucleic acids which preferably consist of RNA which basically comprises two moieties. The first moiety shows a catalytic activity whereas the second moiety is responsible for the specific interaction with the target nucleic acid. Upon interaction between the target nucleic acid and the second moiety of the ribozyme, typically by hybridisation and Watson-Crick base pairing of essentially complementary stretches of bases on the two hybridising strands, the catalytically active moiety may become active which means that it catalyses, either intramolecularly or intermolecularly, the target nucleic acid in case the catalytic activity of the ribozyme is a phosphodiesterase activity. Subsequently, there may be a further degradation of the target nucleic acid which in the end results in the degradation of the target nucleic acid as well as the protein derived from the said target nucleic acid due to a lack of newly synthesized protein corresponding to the target nucleic acid and a turn-over of prior existing respective protein. Ribozymes, their use and design principles are known to the one skilled in the art, and, for example described in Doherty and Doudna (Ribozym structures and mechanism. Annu ref. Biophys. Biomolstruct. 2001; 30: 457-75) and Lewin and Hauswirth (Ribozyme Gene Therapy: Applications for molecular medicine. 2001 7: 221-8).

In a further embodiment the cargo molecule is an antisense molecule. The use of antisense oligonucleotides for the manufacture of a medicament and as a diagnostic agent, respectively, is based on a similar mode of action as the one of siRNA molecules and ribozymes. Basically, antisense oligonucleotides hybridise based on base complementarity, with a target RNA, preferably with an mRNA, thereby activate RNase H. RNase H is activated by both phosphodiester and phosphorothioate-coupled DNA. Phosphodiester-coupled DNA, however, is rapidly degraded by cellular nucleases with the exception of phosphorothioate-coupled DNA. These resistant, non-naturally occurring DNA derivatives do not inhibit RNase H upon hybridisation with RNA. In other words, antisense polynucleotides are only effective as DNA RNA hybrid complexes. Examples for this kind of antisense oligonucleotides are described, among others, in U.S. Pat. No. 5,849,902 and U.S. Pat. No. 5,989,912. In other words, based on the nucleic acid sequence of the respective target molecule, either from the target protein from which a respective nucleic acid sequence may in principle be deduced, or by knowing the nucleic acid sequence as such, particularly the mRNA, suitable antisense oligonucleotides may be designed base on the principle of base complementarity.

Particularly preferred are antisense-oligonucleotides which have a short stretch of phosphorothioate DNA (3 to 9 bases). A minimum of 3 DNA bases is required for activation of bacterial RNase H and a minimum of 5 bases is required for mammalian RNase H activation. In these chimeric oligonucleotides there is a central region that forms a substrate for RNase H that is flanked by hybridising “arms” comprised of modified nucleotides that do not form substrates for RNase H. The hybridising arms of the chimeric oligonucleotides may be modified such as by 2′-O-methyl or 2′-fluoro. Alternative approaches used methylphosphonate or phosphoramidate linkages in said arms. Further embodiments of the antisense oligonucleotide useful in the practice at least some embodiments of the present invention are P-methoxyoligonucleotides, partial P-methoxyoligodeoxyribonucleotides or P-methoxyoligonucleotides.

In a further embodiment the cargo molecule is an aptamer or a spiegelmer. Aptamers are D-nucleic acids which are either single stranded or double stranded and which specifically interact with a target molecule. The manufacture or selection of aptamers is, e.g., described in European patent EP 0 533 838. Basically the following steps are realized. First, a mixture of nucleic acids, i.e. potential aptamers, is provided whereby each nucleic acid typically comprises a segment of several, preferably at least eight subsequent randomised nucleotides. This mixture is subsequently contacted with the target molecule whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture having an increased ratio of nucleic acids specifically binding to the target from which the final binding nucleic acid is then optionally selected. These specifically binding nucleic acid(s) are referred to aptamers. At any stage of the method for the generation or identification of the aptamers samples of the mixture of individual nucleic acids may be taken to determine the sequence thereof using standard techniques. It is within at least some embodiments of the present invention that the aptamers may be stabilized such as, e.g., by introducing defined chemical groups which are known to the one skilled in the art of generating aptamers. Such modification may for example reside in the introduction of an amino group at the 2′-position of the sugar moiety of the nucleotides. Aptamers are currently used as therapeutical agents.

According to at least some embodiments of the present invention, the selected or generated aptamers may be used for target validation. The generation or manufacture of spiegelmers which may be used or generated according to at least some embodiments of the present invention directed against a target molecule, is based on a similar principle. The manufacture of spiegelmers is described in the international patent application WO 98/08856. Spiegelmers are L-nucleic acids, which means that they are composed of L-nucleotides rather than aptamers which are composed of D-nucleotides as aptamers are. Spiegelmers are characterized by the fact that they have a very high stability in biological system and, comparable to aptamers, specifically interact with the target molecule against which they are directed. In the purpose of generating spiegelmers, a heterogonous population of D-nucleic acids is created and this population is contacted with the optical antipode of the target molecule, i.e. with the D-enantiomer of the naturally occurring L-enantiomer of the target molecule. Subsequently, those D-nucleic acids are separated which do not interact with the optical antipode of the target molecule. However, those D-nucleic acids interacting with the optical antipode of the target molecule are separated, optionally determined and/or sequenced and subsequently the corresponding L-nucleic acids are synthesized based on the nucleic acid sequence information obtained from the D-nucleic acids. These L-nucleic acids which are identical in terms of sequence with the aforementioned D-nucleic acids interacting with the optical antipode of the target molecule, will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

In a further embodiment the cargo molecule is a short double-stranded oligodesoxynucleotide acting as a decoy molecule by specifically binding to transcription factors inside the cell. These decoy molecules are said to be taken up efficiently by cells without a need for specific carriers or delivery agents. According to at least some embodiments of the present invention, the efficiency and cytoplasmic delivery and/or tissue specificity may be further enhanced by conjugation to a CPP.

In a further embodiment the cargo molecule is an antibody. The manufacture of an antibody is known to the one skilled in the art and, for example, described in Harlow, E., and Lane, D., “Antibodies: A Laboratory Manual,” Cold Spring Harbor Laboratory, Cold Spring Harbor, N.Y., (1988). Preferably, monoclonal antibodies may be used in connection with at least some embodiments of the present invention which may be manufactured according to the protocol of Köhler and Milstein and further developments based thereon. Antibodies as used herein, include, but are not limited to, complete antibodies, antibody fragments or derivatives such as Fab fragments, Fc fragments and single-stranded antibodies, as long as they are suitable and capable of binding to protein kinase N beta. Apart from monoclonal antibodies also polyclonal antibodies may be used and/or generated. The generation of polyclonal antibodies is also known to the one skilled in the art and, for example, described in Harlow, E., and Lane, D., “Antibodies: A Laboratory Manual,” Cold Spring Harbor Laboratory, Cold Spring Harbor, N.Y., (1988). Preferably, the antibodies used for therapeutical purposes are humanized or human antibodies as defined above.

The antibodies which may be used according to at least some embodiments of the present invention may have one or several markers or labels. Such markers or labels may be useful to detect the antibody either in its diagnostic application or its therapeutic application. Preferably the markers and labels are selected from the group comprising avidine, streptavidine, biotin, gold and fluorescein and used, e.g., in ELISA methods. These and further markers as well as methods are, e.g. described in Harlow, E., and Lane, D., “Antibodies: A Laboratory Manual,” Cold Spring Harbor Laboratory, Cold Spring Harbor, N.Y. (1988).

It is also within at least some embodiments of the present invention that the label or marker exhibits an additional function apart from detection, such as interaction with other molecules. Such interaction may be, e.g., specific interaction with other compounds. These other compounds may either be those inherent to the system where the antibody is used such as the human or animal body or the sample which is analysed by using the respective antibody. Appropriate markers may, for example, be biotin or fluoresceine with the specific interaction partners thereof such as avidine and streptavidine and the like being present on the respective compound or structure to interact with the thus marked or labelled antibody.

In a further embodiment the cargo molecule is a target specific binding peptide. Such peptides may be generated by using methods according to the state of the art such as phage display. Basically, a library of peptide is generated, such as in form of phages, and this kind of libraries is contacted with the respective target molecule. Those peptides binding to the target molecule are subsequently removed, preferably as a complex with the target molecule, from the respective reaction. It is known to the one skilled in the art that the binding characteristics, at least to a certain extent, depend on the particularly realized experimental set-up such as the salt concentration and the like. After separating those peptides binding to the target molecule with a higher affinity or a bigger force, from the non-binding members of the library, and optionally also after removal of the target molecule from the complex of target molecule and peptide, the respective peptide(s) may subsequently be characterised. Prior to the characterisation optionally an amplification step is realized such as, e.g. by propagating the peptide coding phages. The characterisation preferably comprises the sequencing of the target binding peptides. Basically, the peptides are not limited in their lengths, however, preferably peptides having a lengths from about 8 to 20 amino acids are preferably obtained in the respective methods. The size of the libraries may be about 10² to 10¹⁸, preferably 10⁸ to 10¹⁵ different peptides, however, is not limited thereto.

A particular form of target binding peptides are the so-called “anticalines” which are, among others, described in German patent application DE 197 42 706.

Production of CPPs

According to at least some embodiments of the present invention, CPPs may optionally be produced according to any conventional means, including but not limited to chemical synthetic methods and recombinant biological methods.

According to at least some embodiments the present invention is related to a nucleic acid coding for a CPP peptide, optionally with a signal peptide as is known in the art. Such nucleic acid can be easily derived by the ones skilled in the art based on the amino acid sequence of the peptide and the genetic code. It will be acknowledged that depending on the host organism the particular sequence can be adapted to the codon usage of the respective host organism. According to further embodiments of the present invention, the nucleic acids sequences encoding the CPP peptides SEQ ID NOs: 1-6 are depicted in SEQ ID NOs: 33-38 respectively.

According to at least some embodiments the present invention is related to a nucleic acid coding for a peptide, whereby said peptide consists of a CPP peptide according to at least some embodiments of the present invention and a further peptide or protein, and whereby said protein is generally referred to as fusion peptides/proteins. According to protocols known to those skilled in the art this nucleic acid may either serve the expression and purification of recombinant proteins, in which at least one part comprise the CPP peptide(s), and at least one other part as the further peptide or protein. In a further embodiment the nucleic acid coding for the further peptide or protein which serves as vaccination moiety and is fused to the nucleic acid coding for the CPP peptide.

According to at least some embodiments, the present invention is related to a fusion protein as defined herein and more particularly to a fusion protein encoded by a nucleic acid coding for a fusion protein according to at least some embodiments of the present invention.

According to at least some embodiments the present invention is related to a composition comprising any of a complex, fusion molecule and/or conjugate according to at least some embodiments of the present invention, a composition comprising a peptide according to at least some embodiments of the present invention, a composition comprising a nucleic acid coding for a peptide according to at least some embodiments of the present invention, a composition comprising a peptide according to at least some embodiments of the present invention and a cargo molecule, a composition comprising a fusion protein, as DNA or RNA binding domain, according to at least some embodiments of the present invention and a composition comprising a nucleic acid coding for such fusion. It is within the skills of the one of the art that such compositions according to at least some embodiments of the present invention may comprise one or several of the peptides according to at least some embodiments of the present invention, one or several of the nucleic acids according to at least some embodiments of the present invention, and/or one or several of the cargo molecules. In connection therewith it is preferred that the term “several” means several different species of the respective compounds or molecules. It will be well acknowledged by the ones skilled in the art that the composition typically comprises a multitude of the individual species of the peptide according to at least some embodiments of the present invention, of the nucleic acid coding for such peptide and/or the of the cargo molecule. In connection therewith it is to be understood that any of the cargo molecules described herein can be used.

### Peptide and Cargo Bond

According to at least some embodiments of the present invention the CPP peptide is attached to a cargo molecule. Such cargo molecule may be any cargo molecule as defined herein. The attachment can be either a fusion or a complex as defined above, such that the attachment may optionally be covalent or non-covalent, respectively, comprising at least one peptide according to at least some embodiments of the present invention and at least one cargo molecule. It is also within in the present invention that the attachment comprises more than one peptide according to at least some embodiments of the present invention, i.e. a plurality of such peptides, whereby the plurality of the peptides may comprise a plurality of the same or of different peptides. Also, the attachment according to at least some embodiments of the present invention may also comprise more than one cargo molecule, whereby the plurality of the cargo molecules may comprise a plurality of the same or of different cargo molecules.

In one embodiment, the attachment between the peptide(s) according to at least some embodiments of the present invention and the cargo molecule(s) is formed by covalent bonds. Such covalent bonds are preferably formed between either suitable reactive group of the peptide and the cargo and more preferable between a terminus of the peptide according to the present at least some embodiments of the invention and the cargo molecule(s). Depending on the chemical nature or the cargo molecules, the moiety, group or radical with which such covalent bond is formed varies and it is within the skills of a person of the art to create such bond.

In one embodiment, the covalent bond may be an amide bond formed between the carboxy group of the C-terminal amino acid of a peptide according to at least some embodiments of the present invention and the alpha amino group of the N-terminal amino acid of a peptide constituting a cargo molecule or vice versa.

In another embodiment, the covalent bond between the CPP and the cargo molecule can comprise a linker as described above.

Alternatively, the attachment can be formed based on non-covalent bond(s). Such non-covalent bonds can be ionic bonds, hydrogen bonds or hydrophobic interaction or a combination of such bonds. In one embodiment such non-covalent bonds may be formed by a stretch of lysine residues, attached by covalent bonds to a peptide according to at least some embodiments of the present invention and the phosphate backbone of a oligonucleotide. Preferably the stretch of lysine consists of about 5 to 15 lysine residues.

Alternatively, the attachment can be formed by encapsulation of any composition of a CPP and a cargo molecule inside a micelle, micro/nano-particle, polymer related or lipid like materials.

The cargo molecules are, in principle, not limited with regard to size, chemical nature and/or function. In accordance therewith the cargo molecule may be selected from the group consisting of but not limited to nucleic acids, peptides, lipids, carbohydrates, nano- and micro-particles and combinations thereof.

Alternatively, if the cargo is an oligonucleotide such as siRNA, miRNA, DNA or antisense, the attachment can be formed by a DNA binding domain, and more specifically double stranded RNA binding domain to a CPP dsRNA binding domain bind to siRNAs with high avidity, masking the siRNA negative charge (Eguchi A, et al., Nat Biotechnol. 2009 June; 27(6):567-71).

### Tissue Specific Delivery

In order to avoid non-specific internalization of CPPs before finding a target cell in vivo, the invention in at least some embodiments relates to a CPP-complex, fusion and/or conjugate, which is designed through new variation of the enzyme-prodrug strategy, in such a way that the cell penetration-active structure of the CPPs is modified so as to support the specific binding of a peptide part of said CPP to a cell/tissue or organ specific receptor/marker, or the cleavage of said CPP-complex, fusion and/or conjugate by a protease secreted by the target cell/tissue or organ, releases the CPP from conformational discrimination or otherwise permits the CPP to achieve cell penetration at the desired location(s), with at least reduced non-specific penetration.

The term “enzyme-prodrug strategy/therapy” as used herein is used to define a specific approach to delivering a drug, which is focused on the development of amino-acid or nucleic-acid prodrugs, which, before or after delivery, require activation by tissue, organ and/or cell-selective enzymes. The differences in selectivity can derive either from a rapid removal of the released drug from the target tissue/organ/cell, or from cleavage in non-target tissue and insufficient transport across the cell to the enzyme site.

As used herein, the term “enzyme-prodrug strategy/therapy” is additionally used to describe the above revealed method of delivering a drug, wherein the drug itself or its transporter CPP is rendered non-cell-penetrating in order to avoid non-specific internalisation of CPPs before finding a target cell in vivo, and wherein only the binding event of a peptide part of said CPP to a cell/tissue or organ specific receptor/marker, or the cleavage of said CPP-complex, fusion and/or conjugate by a protease secreted by the target cell/tissue or organ, releases the CPP from conformational discrimination, whereupon it can penetrate the target cell.

According to at least some embodiments the present invention relates to a CPP-complex, fusion and/or conjugate which is designed so that the cell penetration-active structure of the CPPs is modified until the detachment or cleavage of said CPP-complex, fusion and/or conjugate by any external physical induction, such as heat induction related and ultrasonic wave induction onto a certain tissue, organ or defined location in a human body. In one example, the means for producing ultrasonic waves operate at a frequency in the range of 20 kHz to 200 kHz. According to further specific embodiment, the conjugation between the CPP according to at least some embodiments of this invention and the CPP-complex, fusion and/or conjugate is a linker which is destabilized by heat or ultrasonic wave induction.

In still further embodiment, the CPP peptides according to at least some embodiments of the present invention, is coupled to a moiety that intramolecularly masks the CPP and prevents the CPP from acting as a cell penetrating protein, such a strategy is called pro-drug or more specifically an enzyme pro-drug strategy. An enzymatically cleavable bond is incorporated between the CPP and the masking moiety. For example, such an intramolecular masking approach has been described for the targeting of a fluorophore attached to the CPP nonaarginine. The nonaarginine CPP was linked to a hexaglutamic acid stretch via a peptide linker corresponding to the cleavage site for matrix metalloproteinases 2 and 9. These proteases are secreted by tumor cells in high concentrations. Secretion of proteases selectively cleaves the CPP-mask construct in the vicinity of tumor cells, thereby enabling the efficient uptake of the CPP-cargo construct into the tumor cells (T. Jiang et al., Proc. Natl. Acad. Sci, USA, 101, 17867-17872, 2004). Accordingly, this embodiment represents an effective targeting or delivery means for tumor specific targeting and/or delivery or for any organ/tissue specific delivery.

According to at least some embodiments of the present invention provides a CPP-complex, fusion and/or conjugate, designed so that the cell penetration-active structure of the CPPs is modified by a covalently or non-covalently attached entity, preferably a molecule with certain affinity to the CPP, such as small molecule, lipid like molecule, oligonucleotide or a polymer construct and more preferably a peptide.

According to at least some embodiments of the present invention, the CPP peptides according to at least some embodiments of the present invention are delivered to a specific type of cells or tissue or organ comprising such specific type of cells. In a preferred embodiment such specific delivery is mediated through a targeting moiety or targeting molecule which are, in their entirety, also referred to herein as the targeting entity.

In a further embodiment, such targeting moiety is either part of the CPP peptides according to at least some embodiments of the present invention or part of the cargo molecule. Alternatively or additionally, such targeting moiety is part of the complex, fusion and/or conjugate, or composition according to at least some embodiments of the present invention.

For example, sugars, such as monosaccharides (ex: glucose, galactose, glucosamine or galactosamine), oligosaccharides, polysaccharides, or their analogs, as well as some oligonucleotides, or some organic molecules such as folate, can be used as targeting entities. Without wishing to be limited by a single hypothesis, their targeting activity is due to the fact that they are recognized as ligands by some receptors which are over-expressed at the surface of cells in the zone of interest.

Other non-limiting examples of the targeting entity include one or more of peptides, proteins, including antibodies, antibody fragments, single chain antibodies, aptamers, spiegelmers and ligands binding cell surface receptors. It will be acknowledged by the ones skilled in the art that in principle a partner moiety or molecule of any combination of interaction partners can be used which provides for a targeting, as a targeting entity. This includes the use of ligands to receptors which are expressed and more particularly overexpressed on a distinct cell type or ligands or molecules which are expressed and more particularly overexpressed on a distinct cell type. In the latter case a particularly prominent interaction partner thereof which acts as a targeting entity is selected from the group comprising antibodies, aptamers, spiegelmers, highly specific binding peptides, and anticalines. This kind of interaction partners and their specificity for a particular type of cell are known to the ones skilled in the art. Among others, the ErbB2 protein is specific for breast cancer cells. Accordingly, an antibody directed thereagainst is a suitable targeting entity.

In a further embodiment, the targeting moiety is included in or on the particles described herein which may be used as cargo molecules. Due to the size of such particles, the use of a more bulky targeting entity such as an antibody is preferable in connection with such embodiment.

According to at least some embodiments, the present invention relates to cell type or tissue specific targeted CPPs which can further be modified by a non-covalent intermolecular interaction or a covalent conjugation with a homing entity, such as a part of a receptor targeting sequence, or homing peptide discovered by phage display which is further elaborated below, or any other technique or method which produces homing characteristic molecules.

According to at least some embodiments the present invention provides a composition comprising such a homing entity-CPP construct and a cargo molecule of any type mentioned above.

### Pharmaceutical Compositions and Administration

The present invention, in at least some embodiments, also relates to a pharmaceutical composition comprising a peptide according to at least some embodiments of the invention or a variant thereof or a complex, or a fusion or a conjugate comprising the same, in admixture with pharmaceutically acceptable auxiliaries, and optionally other therapeutic agents. The auxiliaries must be “acceptable” in the sense of being compatible with the other ingredients of the composition and not deleterious to the recipients thereof.

The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions according to at least some embodiments of the present invention may be manufactured by processes well known in the art; e.g., by means of conventional mixing, dissolving, granulating, dragee-making, levigating, emulsifying, encapsulating, entrapping or lyophilizing processes.

Pharmacological compositions for use in accordance with the present at least some embodiments of the invention, may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Suitable routes of administration of a peptide or pharmaceutical composition comprising a peptide according to at least some embodiments of the invention are oral, rectal, pulmonary (e.g. inhalation), nasal, topical (including transdermal, buccal and sublingual), vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intravenous and intradermal) administration or administration via an implant. In a specific embodiment, a peptide or a pharmaceutical composition comprising a peptide according to at least some embodiments of the invention can be administered intravenously.

The exact dose and regimen of administration of a peptide or pharmaceutical composition comprising a peptide according to at least some embodiments of the invention will necessarily be dependent upon the therapeutic effect to be achieved (e.g. treatment of an auto-immune disease) and may vary with the particular compound, the route of administration, and the age and condition of the individual subject to whom the medicament is to be administered.

For injection, the compounds according to at least some embodiments of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Formulation that promote penetration of the epidermis are known in pharmacology, and can find use in the treatment of many skin conditions, such as, but not limited to, psoriasis and fungal infections. Formulations that promote penetration of the epidermis and underlying layers of skin are also known, and can be used to apply compositions according to at least some embodiments of the present invention to, for example, underlying muscle or joints. In some preferred therapeutic embodiments, formulation comprising compositions according to at least some embodiments of the present invention that deliver compounds for alleviation rheumatoid or osteo-arthritis can be administered by applying a cream, ointment or gel to the skin overlying the affected joint.

Oral and parenteral administration may be used where the peptide and/or complex and/or fusion molecule and or conjugate is made stable enough to weather the harsh proteolytic environment of the gut. If so, the composition according to at least some embodiments of the present invention can be formulated readily by combining the active compounds with pharmaceutically acceptable carriers well known in the art. Such carriers enable the compounds according to at least some embodiments of the invention to be formulated as tablets, pills, dragees, capsules, liquids, gels, syrups, slurries, suspensions and the like, for oral ingestion by a patient to be treated. Pharmacological preparations for oral use can made with the use of a solid excipient, optionally grinding the resulting mixture, and processing the mixture of granules, after adding suitable auxiliaries, if desired, to obtain tablets of dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragée cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solution, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may taken in the form of tablets or lozenges formulated in conventional manner. For the small peptides and complexes, fusion molecules and/or conjugates according to at least some embodiments of the invention, this may prove useful.

For administration by inhalation, the composition according to at least some embodiments of the present invention are conveniently delivered in the form of an aerosol spray presentation from pressurized packs or a nebulizer, with the use of a suitable propellant, e.g., dichlorodifluoromethane, trichlorofluoromethane, dichloro-tetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The composition according to at least some embodiments of the present invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include aqueous solutions of the compositions in water soluble form. Additionally, suspensions of the compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-fee water, before use.

The compositions may also be formulated in rectal compositions such as suppositories or retention enemas, e.g., containing conventional suppository bases such as cocoa butter or other glycerides.

In addition to the formulations described previously, the composition according to at least some embodiments of the present invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil), or as part of a solid or semi-solid implant that may or may not be auto-degrading in the body, or ion exchange resins, or one or more components of the composition can be formulated as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use according to at least some embodiments of the present invention include compositions wherein the active ingredients are contained in an amount effective to achieve its intended purpose. More specifically, a therapeutically effective amount means an amount of compound effective to prevent, alleviate or ameliorate symptoms of disease or prolong the survival of the subject being treated.

Determination of a therapeutically effective amount is within the capability of those skilled in the art, especially in light of the detailed disclosure provided herein.

For any compound used in the methods according to at least some embodiments of the invention, the therapeutically effective amount or dose can be estimated initially from cell culture assays. For example, a dose can be formulated in animal models to achieve a circulating concentration range that includes the IC₅₀ as determined in cell culture (where inhibitor molecules are concerned). Such information can be used to more accurately determine useful doses in humans.

Toxicity and therapeutic efficacy of a composition according to at least some embodiments of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage may vary within this range depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

The amount of composition administered will, of course, be dependent on the subject being treated, on the subject's weight, the severity of the affliction, the manner of administration and the judgment of the prescribing physician.

A pharmaceutical composition that comprises a peptide of a composition according to at least some embodiments of the present invention can be supplied such that the peptide and one or more of the cargo molecules are in the same container, either in solution, in suspension, or in powder form. The peptide according to at least some embodiments of the present invention can also be provided separately from one or more of the cargo molecules, and can be mixed with one or more of the cargo molecules prior to administration. Various packaging options are possible and known to the ones skilled in the art, depending, among others, on the route and mechanism of administration. For example, where the peptide according to the present is supplied separately from one or more of the cargo molecules, the compositions may, if desired, be presented in a pack having more than one chamber, and in which a barrier can be ruptured, ripped, or melted to provide mixing of the peptide according to at least some embodiments of the present invention with the cargo molecule. Alternatively, two separately provided elements can be mixed in a separate container, optionally with the addition to one or more other carriers, solutions, etc. One or more unit dosage forms containing the cargo molecule can be provided in a pack. The pack or dispenser device may be accompanied by instructions for administration. Compositions comprising a compound according to at least some embodiments of the invention formulated in a compatible pharmaceutical carrier may also be prepared, placed in an appropriate container, and labeled for treatment of an indicated condition. Suitable conditions indicated on the label may include any disease which may be treated or prevented or diagnosed using the compositions according to at least some embodiments of the present invention. In particular, the invention is ideally suited to gene therapy.

In at least some embodiments, the invention further includes a pharmaceutical composition, as hereinbefore described, in combination with packaging material, including instructions for the use of the composition for a use as hereinbefore described.

Uses and Methods

According to at least some embodiments the present invention is related to the use of any of the compositions or constituents thereof described herein as transfection agent, as a medicament, and/or as a diagnostic agent. In case the composition is used as a medicament or as a pharmaceutical composition, preferably the cargo molecule is a pharmaceutically active agent. Such pharmaceutically active agents may be a chemotherapeutic agents, such as for example, daunorubicin, doxorubicin or peptides interfering with molecular interactions inside the cell, siRNA drugs, or any of the molecules described herein as cargo molecules, whereby preferably said cargo molecules are pharmaceutically active or a pre-forms of such pharmaceutically active molecules. In case the composition is used as a diagnostic agent, preferably the cargo molecule is a diagnostic marker.

As mentioned hereinabove the CPP peptides according to at least some embodiments of the present invention, and/or or nucleic acid sequence encoding same, and/or pharmaceutical compositions comprising same, may be used to deliver cargo molecules inside the cell, thus deliver a cargo to the cytoplasm, nucleolus or any organelle within the cell. CPP peptides, variants thereof and/or complexes, and/or fusion molecules and/or conjugates comprising same can be used for manufacture of pharmaceutical compositions for treating of a wide range of conditions, disorders and diseases. CPP peptides, variants thereof and/or complexes, and/or fusion molecules and/or conjugates comprising same can be further used for manufacture of diagnostic compositions for diagnosing of a wide range of conditions, disorders and diseases. The CPPs and/or variants thereof and/or complexes and/or fusion molecules and/or conjugates and/or pharmaceutical compositions comprising same can be used for treating and/or preventing of a wide range of conditions, disorders and diseases. The CPPs and/or variants thereof and/or complexes and/or fusion molecules and/or conjugates and/or pharmaceutical compositions comprising same can be used for diagnosing of a wide range of conditions, disorders and diseases.

The subject according to at least some embodiments of the present invention is optionally and preferably a mammal, preferably a human which is diagnosed with one of the disease, disorder or conditions described hereinabove, or alternatively is predisposed to at least one of the diseases, disorders or conditions described hereinabove.

The CPP peptides according to at least some embodiments of the present invention, and/or or nucleic acid sequence encoding same, and/or pharmaceutical compositions comprising same, can be administered together with another agent, whether sequentially (in either order) or simultaneously. It will be appreciated that treatment of cancer according to at least some embodiments of the present invention may be combined with other treatment methods known in the art (i.e., combination therapy). Thus, treatment of malignancies using the agents according to at least some embodiments of the present invention may be combined with, for example, radiation therapy, antibody therapy and/or chemotherapy.

In yet another embodiment, CPP peptides according to at least some embodiments of the present invention, and/or or nucleic acid sequence encoding same, and/or compositions comprising same optionally may be used to target compounds (e.g., therapeutic agents, labels, cytotoxins, radiotoxins immunosuppressants, etc.) to specific cells.

According to at least some embodiments the present invention provides methods for localizing ex vivo or in vivo cells displaying the CPP peptides according to at least some embodiments of the present invention, and/or or nucleic acid sequence encoding same, and/or pharmaceutical compositions comprising same (e.g., with a detectable label, such as a radioisotope, a fluorescent compound, an enzyme, or an enzyme co-factor).

### Diagnostic Uses

Molecular imaging of intracellular and intranuclear targets, by techniques such as SPECT, PET, optical imaging, and MRI, relies heavily on the delivery of contrast agents to the cytoplasm and/or nuclei of the target tissue. Therefore, the number of applications in molecular imaging of intracellular targets has remained relatively low, because of the effective barrier presented by the cell membrane. One of the key strategies to overcome this challenge is the introduction of membrane-transducing peptides in the design of new contrast agents.

According to at least some embodiments of the present invention the CPP peptides, variants thereof and/or polynucleotides encoding same and/or complexes and/or fusion molecules and/or conjugates and/or compositions comprising the same are used as diagnostic markers for diagnosis of a wide range of conditions, disorders and diseases.

In at least some embodiments the present invention provides CPP peptides, variants thereof and/or polynucleotides encoding same and/or complexes and/or fusion molecules and/or conjugates and/or compositions comprising same, which may optionally be used as diagnostic markers for in vivo imaging. Molecular in vivo imaging using these diagnostic markers could be performed in conjuction with other imaging modalities as CT and MRI which capture body anatomy and overlap it with the in-vivo marker distribution.

In at least some embodiments of the present invention, the diagnostic methods are selected from any in vivo imaging methods known in the art, including, but not limited to, radioimaging, positron emission tomography (PET), single photon emission computer tomography (SPECT), magnetic resonance imaging (MRI), Ultra Sound, Optical Imaging, Computer Tomography, radioimmunoassay (RIA), ELISA, slot blot, competitive binding assays, fluorimetric imaging assays, Western blot, FACS, and the like. The diagnostic assays can be qualitative or quantitative.

According to at least some embodiments, the present invention provides a method for diagnosing a specific condition, disorder and/or disease, such as cancer, in a patient, comprising administering to a patient any one of the CPPs labeled by a detectable molecule, such as radioactive isotope, fluorophore (such as FAM (Carboxyfluorescein)), etc., followed by imaging the bio distribution of the diagnostic marker and more specifically the accumulation of the diagnostic marker within the relevant tissue and more specifically a tissue with an intracellular target which the diagnostic marker comprising the CPP is directed to.

Non-limiting examples of methods or assays are described below.

Each polypeptide of the present according to at least some embodiments of the present invention can be used alone or in combination with other markers.

The above diagnoses may also optionally include differential diagnosis of the disease to distinguish it from other diseases, including those diseases that may feature one or more similar or identical symptoms.

Radio-Imaging Methods

These methods include but are not limited to, positron emission tomography (PET) and single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer.

Molecular imaging in patients would greatly benefit from generic, rational mechanisms to target contrast agents and therapeutic drugs to diseased tissues, especially tumors.

Neutralizing CPPs

Cellular uptake of CPPs, according to at least some embodiments of the present invention, can be largely blocked by fusing them by means of cleavable linkers to polyanionic sequences, which neutralize the polycations by forming intramolecular hairpins of ≈2-3 kDa, because cleavage of the linkers dissociates the inhibitory polyanions, releasing the polycationic peptides and their cargo to attach to and enter cells. The mechanism is a flexible, modular, amplifying strategy to concentrate imaging and therapeutic agents on and within cells in the immediate vicinity of extracellular cleavage activities, such as matrix metalloproteinases (MMPs) in tumors. An example of well characterized proteases overexpressed by tumors, that can be used as primary initial targets are MMP-2 and MMP-9 (Tao Jiang et al., PNAS, 2004 vol. 101 17867-17872).

In still further example the present invention provides in at least some embodiments use of a CPP peptide which is coupled via a cleavable linker to a neutralizing peptide (i.e. inhibiting cell penetration ability of the CPP). Upon exposure to proteases characteristic of a tumor tissue, the linker is cleaved, dissociating the neutralizing peptide and allowing the CPP to bind to and enter tumor cells, thereby visualizing the tumor.

According to at least some embodiments the present invention is related to a method for the treatment or prevention of a wide range of conditions, disorders and diseases in a patient, comprising the administration of a composition according to at least some embodiments of the present invention.

According to at least some embodiments the present invention is related to a method for diagnosing of a wide range of conditions, disorders and diseases in a patient comprising the administration or use of a composition according to at least some embodiments of the present invention.

According to at least some embodiments the present invention is related to nucleic acid sequences coding for a CPP peptide as described herein, used as a vaccine or part of a vaccine. Preferably, the vaccine comprises a nucleic acid, more preferably an RNA, coding for an antigen which is suitable to elicit an immune response in a host organism, whereby the nucleic acid coding for a CPP peptide according to at least some embodiments of the present invention and the nucleic acid coding for such antigen are administered to said host organism. Such administration may be done separately or in a combined manner. In a further embodiment, the respective nucleic acid may be contained or comprised in a vector, more preferably an expression vector which allows the expression of the nucleic acid(s) in said host organism. The further elements of such vector and in particular of such expression vector are known to the ones skilled in the art and comprise, among others one or several of the following elements: a promoter, an enhancer and a terminator. The antigen is preferably an antigen which is related to disease which is to be treated or prevented by the vaccine according to at least some embodiments of the present invention. In addition, the vaccine may also contain constituents exerting a so-called adjuvants effect and those acting as initiators of T helper cell responses. The invention is further described in the following examples, which are not in any way intended to limit the scope of the inventions as claimed.

### EXAMPLES

### Example 1

### Peptide Synthesis

The synthesis of the peptides corresponding to SEQ ID NOs: 1-6 and 19-23 was performed by both Pepscan (Pepscan Therapeutics Lelystad The Netherlands) and AnaSpec Inc. (34801 Campus Drive Fremont, Calif. 94555, USA) and the synthesis of peptides corresponding to SEQ ID NOs: 24-31 was performed by Pepscan. Peptides corresponding to SEQ ID NOs: 24-31 are control and CPPs, covalently conjugated to (klaklak)2 with GG linker at the C-terminus. The identity of the peptides was confirmed by MALDI-TOF mass spectrometry and the purity was determined by analytical HPLC. Peptides with a purity of less than 85% were purified by preparative HPLC. The final purity of all peptides used was >95%. Peptides corresponding to SEQ ID NOs:1-6 and 19-23 were N-terminally labeled with carboxyfluorescein (FAM) and their C-terminus was amidated. Peptides corresponding to SEQ ID NOs:24-32 were amidated on their C-terminus and acetylated on their N-terminus. SEQ ID NOs: 1, 2, 6, 22 and 40 were synthesized again for example 6 with no amidation at their C-terminal (i.e. free) and N-terminally labeled with carboxyfluorescein (FAM).

The amino acid sequence of the peptides is listed in Table 3 (lower case letters describe D-amino acid):

TABLE 3

| Name | SEQ ID NO | sequence |
|---|---|---|
| S22 | 1 | KKTCRRINYCALNK |
| S24 | 2 | KKTLKKLWKKKKRK |
| S36 | 3 | RRRHQRKYRRY |
| S40 | 4 | FLRRRRRFTRQT |
| S67 | 5 | KKLALYLLLAL |
| S8 | 6 | KPSARMLLLKGFGK |
| TAT | 19 | YGRKKRRQRRR |
| Penetratin | 20 | RQIKIWFQNRRMKWKK |
| 9R | 21 | RRRRRRRRR |
| N2 | 22 | VATIKSVSFYTRK |
| N5 | 23 | RPPGFSPFR |
| N2-klaklak | 24 | VATIKSVSFYTRKGG-klaklakk |
| | | laklak |
| R9-klaklak | 25 | RRRRRRRRRGG-klaklakklakl |
| | | ak |
| TAT-klaklak | 26 | YGRKKRRQRRRGG-klaklakkla |
| | | klak |
| S24-klaklak | 27 | KKTLKKLWKKKKRKGG-klaklak |
| | | klaklak |
| S22-klaklak | 28 | KKTCRRINYCALNKGG-klaklak |
| | | klaklak |
| S36-klaklak | 29 | RRRHQRKYRRYGG-klaklakkla |
| | | klak |
| S8-klaklak | 30 | KPSARMLLLKGFGKGG-klaklak |
| | | klaklak |
| klaklak | 31 | klaklakklaklak |
| S40-klaklak | 32 | FLRRRRRFTRQTGG-klaklakkl |
| | | aklak |
| 9R-K | 40 | RRRRRRRRRK |

The peptides were dissolved in double distilled water to concentrations of 0.5 mg/ml. These stock solutions were further diluted in PBS or medium.

### Example 2

### Uptake efficiency of CPPs

### Flow Cytometry.

To determine the efficiency of peptide loading, HeLa cells (ATCC number CCL-2TM (Manassas, Va.) grown in DMEM medium with stabilized glutamine supplemented with 10% fetal calf serum (Biological Industries, Beit Haemek, Israel), were seeded at a density of 20,000 per well in 96 U shape-well plates (Nunc A/S, Roskilde, Denmark) in serum containing DMEM (D-Mem W/O Na Pyr, product number 41965039).

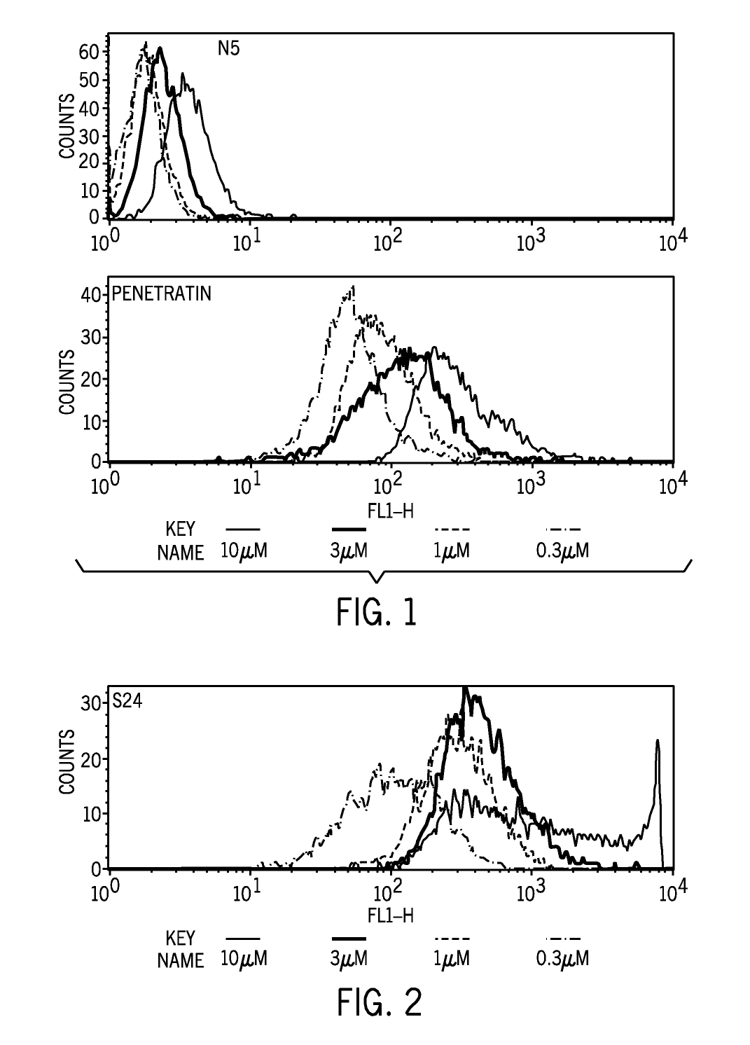

After 24 hours, the cells were washed twice with 300 μL serum free OptiMEM (Reduced Serum Media, buffered with HEPES and sodium bicarbonate and supplemented with hypoxanthine, thymidine, sodium pyruvate, L-glutamine, trace elements and growth factors) medium and incubated in 50 μL serum free OptiMEM, containing peptides in the appropriate concentrations for 1 hour at 37° C. Each condition was tested in triplicates. After incubation, cells were washed with serum free medium, detached by trypsinization for 5 minutes, suspended in ice-cold PBS (phosphate buffered saline) containing 0.1% (w/v) BSA (bovine serum albumin), and measured immediately by flow cytometry (BD FACS Calibur System, Becton Dickinson, Heidelberg, Germany). The fluorescence of approximately 10,000 vital cells was acquired and gated based on sideward and forward scatter. Peptides used as positive controls were Tat(47-57) (SEQ ID NO:19; Jain M, et al., Cancer Res. 2005 Sep. 1; 65(17):7840-6), 9R (SEQ ID NO:21; Mueller J, et al., Bioconjug Chem. 2008 December; 19(12):2363-74) and penetratin (SEQ ID NO:20; Mueller J, et al., Bioconjug Chem. 2008 December; 19(12):2363-74). Negative control peptides N2 (SEQ ID NO:22) and N5 (SEQ ID NO:23) were designed according to Handbook [Cell-Penetrating Peptides, Second Edition, Edited by Ülo Langel, CRC Press 2007, Print ISBN: 978-0-8493-5090-0] and [US Patent Application 20080234183]. The results are presented in FIGS. 1 and 2 and in Table 2.

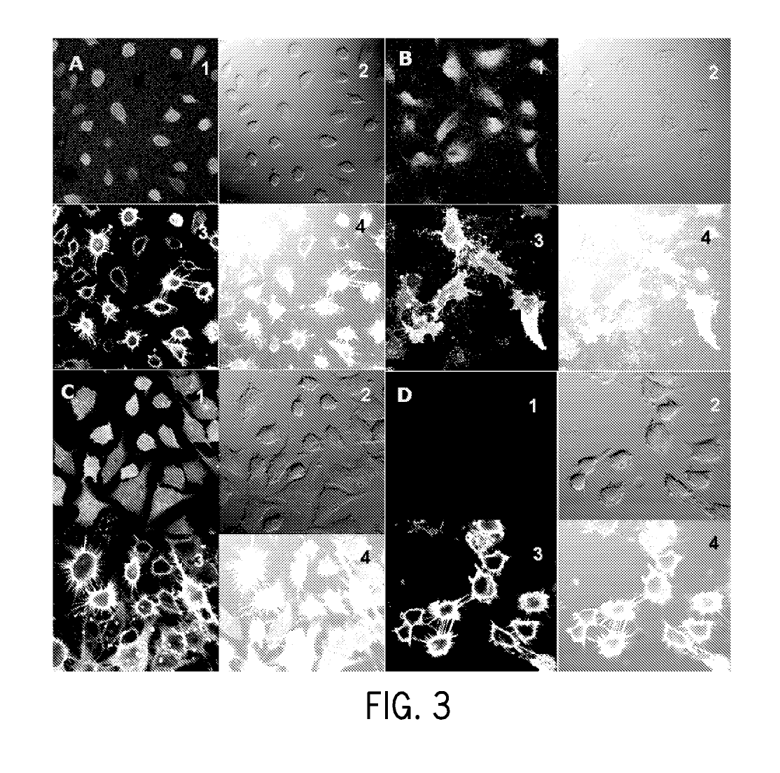

FIG. 1 presents the results of quantification of cellular uptake of two FAM labeled control peptides, N5 and Penetratin (SEQ ID NO:23 and 20), at 37° C. in different doses, by flow cytometry (FACS) analysis. The X axis represents the fluorescent intensity measured and the Y axis represents the number of cells with the intensity measured. For example, at 10 uM concentration, Penetratin is measured with a mean fluorescence of value of approximately 500, indicating high capability of penetration as opposes to N5 in the top graph, where the mean fluorescence at the same concentration is approximately 5. These results are consistent with confocal microscopy results shown in FIG. 3 herein.

FIG. 2 presents the results of quantification of cellular uptake of FAM labeled peptide, S24 (SEQ ID NO:1), at 37° C. by flow cytometry (FACS) analysis in different doses. The X axis represents the fluorescent intensity measured and the Y axis represents the number of cells with the intensity measured. As demonstrated in FIG. 2, S24 illustrates very efficient intracellular uptake in different doses.

Table 4 presents a summary of flow cytometry (FACS) analysis of cellular uptake at 37° C. and concentration of 10 uM of the CPPs (SEQ ID NOs: 1-6). The geometric mean (a quantity calculated by FACS auxiliary software based on measurements such as shown in FIGS. 1 and/or 2 of FL1 intensity is given as the average of a number of experiments, as indicated on the right column. As shown in Table 4, all CPPs show moderate to high cellular uptake as compared to the positive control, Tat(47-57) (SEQ ID NO:19), while the negative control, N5 (SEQ ID NO:23), is shown to have a very low geometric mean value, indicating a low cellular uptake as expected.

TABLE 4

| | Peptide | Geometric mean of FL1 | |
|---|---|---|---|
| SEQ ID | name | intensity | Number of experiments |
| 1 | S22 | 206.4275 | 4 |
| 2 | S24 | 1513.63 | 1 |
| 3 | S36 | 1070.41 | 1 |
| 4 | S40 | 1030.9 | 3 |
| 5 | S67 | 27.9 | 1 |
| 6 | S8 | 52.29 | 1 |
| 19 | Tat(47-57) | 96.95 | 7 |
| 23 | N5 | 8.02 | 8 |

### Confocal Microscopy

To determine qualitative validation of peptide membrane penetration, HeLa cells from 5 ml of confluent T75 flask were seed in 12 well plate at 10×10⁴ cells/mL in 0.5 ml DMEM supplied with 10% Fetal Calf Serum. After 24 h incubation with growth medium, the wells were replaced with Serum Free growth medium for 24 hr, and then the cells were washed with OptiMEM and replaced with 0.5 ml peptide solution at 10 uM for 1 h at 37° C. DiD (DiD-[1,1′-Dioctadecyl-3,3,3′,3′-tetramethylindodicarbocyanine iodide] Lipophilic membrane tracer) (5 ug/ml) was added to each well and incubated for 5 min at 4° C., washed twice with cold PBS and fixed with 3.7% PFA 20′ RT. Cells were rinsed with PBS and mounted on slides for confocal microscopy analysis. The results are presented in FIGS. 3-5.

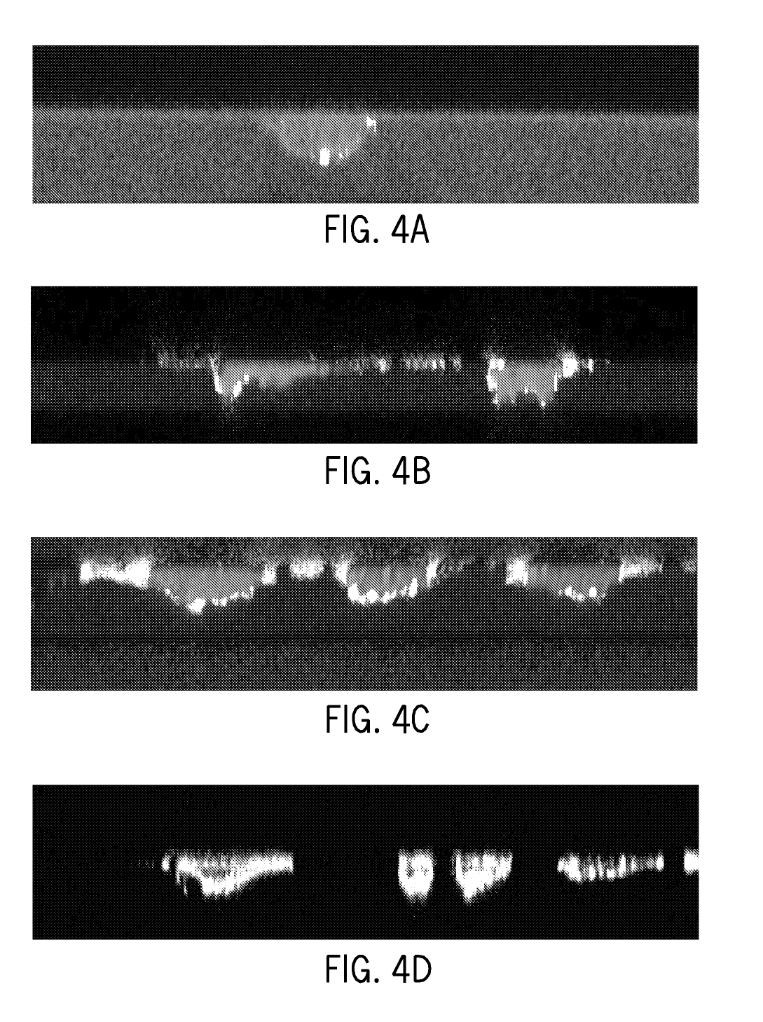

FIG. 3 presents single section confocal microscopy images divided into four quarters A-D where each quarter represents the cellular distribution of the peptide, as follows: A: TAT, B: Penetratin, C: 9R and D: N5. Each such quarter consists of 4 images, 1-4 which are based on the identical image but sampled using different color filters of the confocal microscopy, as follows: sub quarter 1 (i.e. FIG. 3A-1, FIG. 3B-1, FIG. 3C-1 and FIG. 3D-1) is based on a green color filter, which demonstrates the distribution of the FAM labeled peptide inside the cells as dark gray, sub quarter 2 (i.e. FIG. 3A-2, FIG. 3B-2, FIG. 3C-2 and FIG. 3D-2) demonstrates the original image without any filtering, sub quarter 3 (i.e. FIG. 3A-3, FIG. 3B-3, FIG. 3C-3 and FIG. 3D-3) is based on a red color filter, demonstrates the cells membrane staining as bright gray, and sub quarter 4 (i.e. FIG. 3A-4, FIG. 3B-4, FIG. 3C-4 and FIG. 3D-4) demonstrates both red and green original colors as bright and dark gray. All control peptides were labeled with FAM and incubation was done in concentration of 10 uM of peptides and at 37° C. The dark gray colors illustrate the different accumulation of FAM labeled peptides inside the cell (e.g. black color illustrate no accumulation inside the cell as in the negative control (D) and dark gray color illustrate a strong accumulation as in the positive control images, A, B and C). These images demonstrate that the accumulation of peptides occurs inside the cell and not outside or on the surface of the cells. These confocal microscopy results are consistent with FACS analysis results, shown in FIG. 1, and show that positive and negative controls act as expected in this experimental validation system.

FIG. 4 presents the equivalent single cross section images of the 4 control peptides described in FIG. 3, at concentration of 10 uM at 37° C. Tat(47-57) is shown in FIG. 4(A). Penetratin is shown in FIG. 4(B). 9R is shown in FIG. 4(C). Negative control N5 peptide is shown in FIG. 4(D). Single side section images of membrane penetration show the accumulation and distribution of peptides inside cells (e.g. cytoplasm). These results show a strong difference between membrane staining (bright gray), FAM labeled peptide intracellular accumulation (dark gray) and no accumulation (black color).

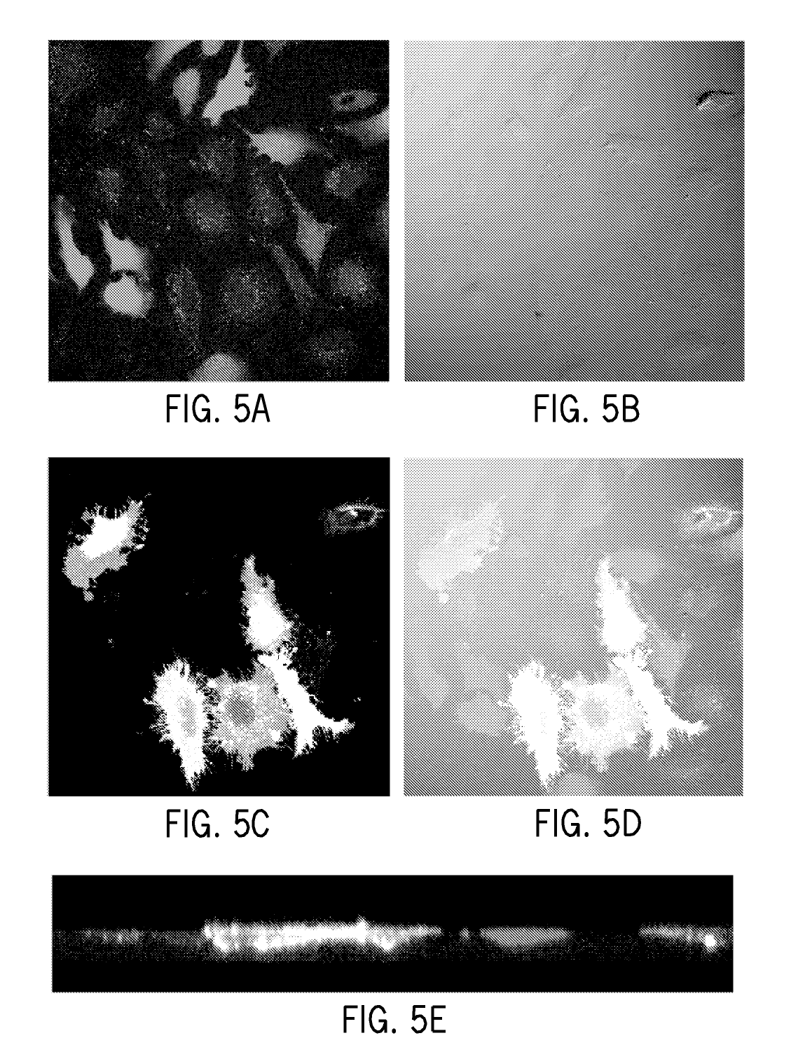

FIG. 5 presents the cellular distribution of peptide S24 (SEQ ID:2) at concentration of 10 uM at 37° C. in equivalence to the description of FIG. 3: quarter 1 is based on a green color filter, which demonstrates the distribution of the FAM labeled peptide inside the cells as dark gray, quarter 2 demonstrates the original image without any filtering, quarter 3 is based on a red color filter, demonstrates the cells membrane staining as bright gray, and quarter 4 demonstrates both red and green original colors as bright and dark gray. The image on the bottom of FIG. 5 is a single side cross section of the cellular distribution of peptide S24, in the same conditions.

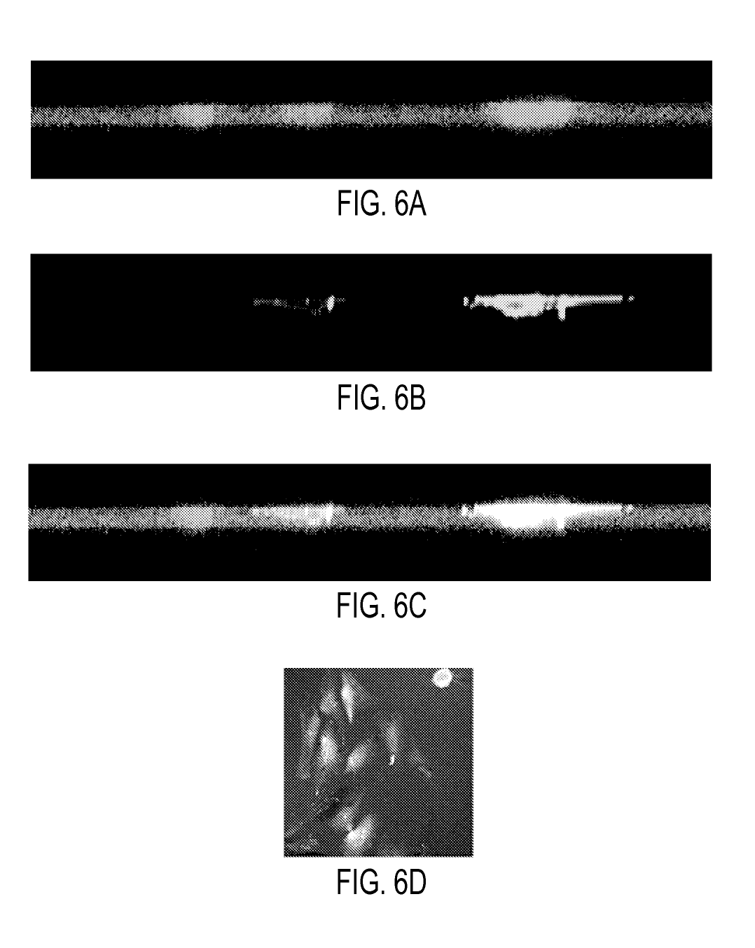

FIGS. 6A-C present single side cross section of confocal microscopy images demonstrating cellular distribution of FAM labeled S22 peptide (SEQ ID NO: 1), (5 uM, 37° C.). FIG. 6A presents intracellular distribution of S22 peptide (dark gray); FIG. 6B presents membrane staining (bright gray); FIG. 6C presents both, S22 peptide cellular distribution and membrane staining. FIG. 6D shows the cellular distribution of S22 peptide (single section, as sub quarters 1 in FIG. 3).

The distribution of the dark gray (originally green fluorescent color) illustrates the strong cell membrane penetration capability of S24 peptide (as shown in FIG. 5) and S22 peptide (as shown in FIG. 6). FACS analysis results were consistent with the confocal microscopy results for these peptides, as it was for the control peptides. The cross and upper single section confocal microscopy images (FIG. 3) emphasis the accumulation of peptides inside the cells and not only on the surface of cells.

### Example 3

### Cargo Delivery by CPPs

In order to examine CPPs capability to deliver into the cell a cargo, the ability of the CPPs to facilitate intracellular cargo delivery of a pro apoptotic peptide—klaklakklaklak (also referred herein as (klaklak)2), was examined. (klaklak)2 is unable to efficiently penetrate cell membranes on its own, and thus using this pro-apoptotic peptide requires efficient intracellular delivery to trigger mitochondrial disruption and mitochondrial-dependent apoptosis. HeLa cells were seeded in DMEM supplemented with 10% fetal Bovine serum (FBS) in 96-well plates, 10,000 cells/well in a total volume of 200 μL 24 hours prior to their treatment with the peptide. Cells were incubated with any one of the CPPs and/or any one of the control peptides (25 and μg/ml) conjugated to pro-apoptotic peptide (klaklak)2 through a short flexible linker of Gly-Gly (as described in the above peptide synthesis section for SEQ ID NOs: 24-32) for 16 hours under tissue culture conditions. Cell viability was then assessed using MTT[3-(4,5-dimethylthiazol-2-yl)-2,5-diphenyl-2H-tetrazo-liumbromide] assay (Catalog number M5655, Sigma, Israel). MTT reagent was added to cells for 4 hours, following cell lysis using DMSO and absorbance was measured at 492 nM. As positive controls (klaklak)2 conjugated to TAT or R9 were used, corresponding to SEQ ID NOs:26 and 25, respectively. As negative controls (klaklak)2 on its own (SEQ ID NO:31), (klaklak)2 conjugated to N5 (SEQ ID NO:25) and peptides not conjugated to (klaklak)2 were used.

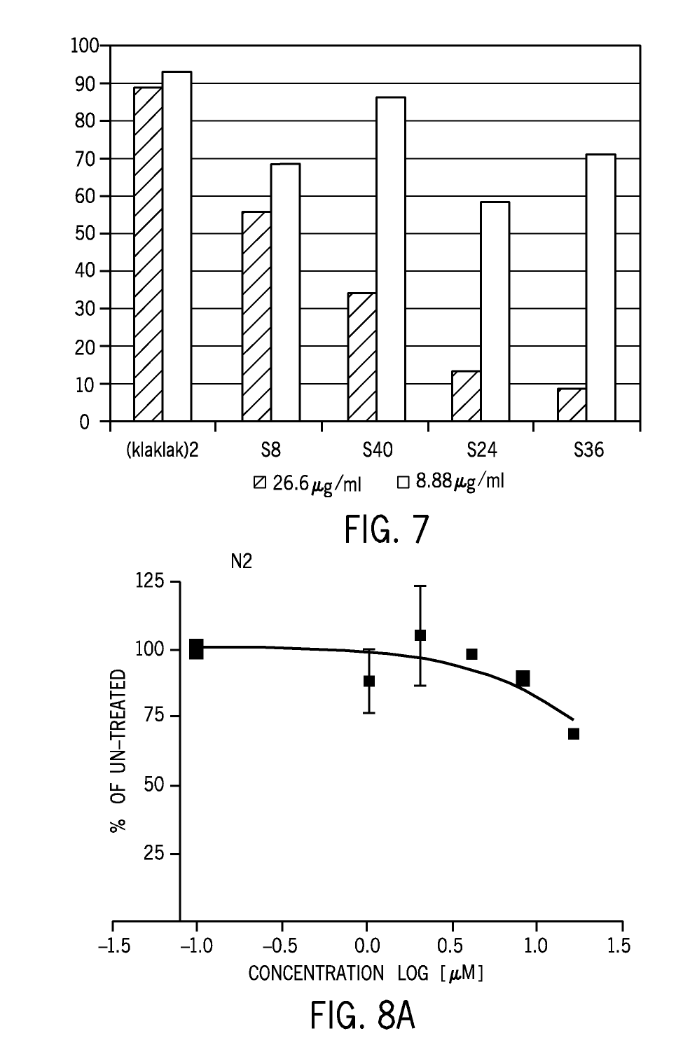

FIG. 7 demonstrates viability of Hela cells as a measurement for (klaklak)2 intracellular delivery in two different concentrations of peptides. As described above, HeLa cells were incubated with the pro-apoptotic (klaklak)2 peptide alone (SEQ ID NO:31) and (klaklak)2 peptide conjugated to any one of S8, S40, S24 and S36 peptides corresponding to SEQ ID NOs: 30, 32, 27 and 29 respectively, and cell viability was then assessed using MTT. The Y axis represents the % of viable cells (normalized by untreated cells and averaged over duplicates). The graph shows a reduction in cell viability for both concentrations for all peptides except the control, (klaklak)2 alone, which as expected demonstrated low penetration ability and thus almost no reduction in cell viability. Incubation of CPPs conjugated to (klaklak)2 (SEQ ID NO: 31), set forth in any one of SEQ ID NOs: 30, 32, 27 and 29, demonstrated reduction in cell viability thus an efficient delivery of the pro-apoptotic peptide into the cell.

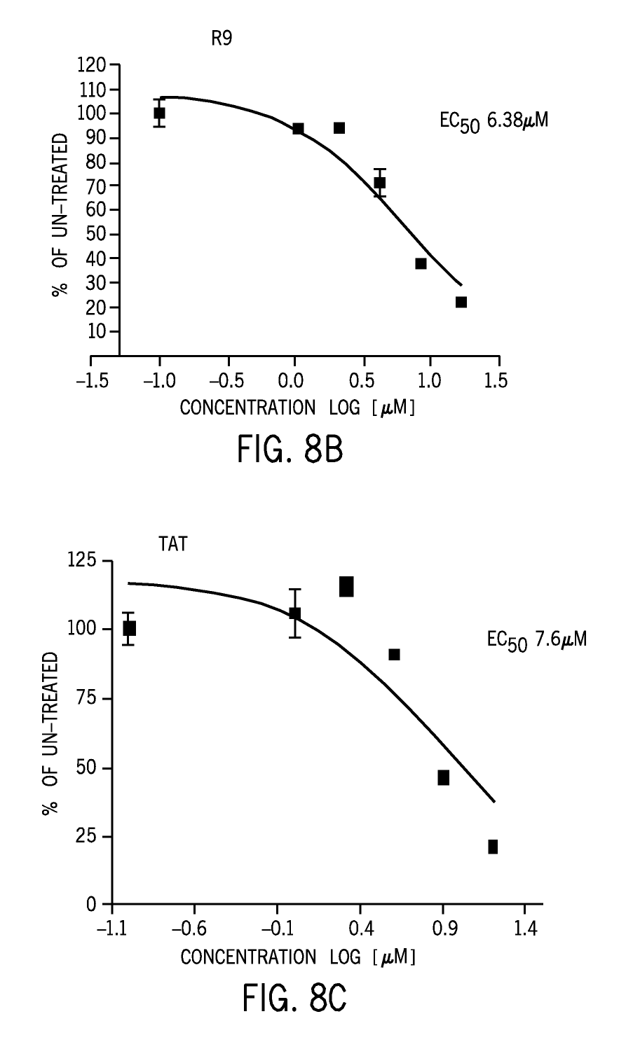

FIG. 8 demonstrates the viability of Hela cells incubated with the pro-apoptotic (klaklak)2 peptide conjugated to control peptides N2 (SEQ ID NO: 24) (FIG. 8A), R9 (SEQ ID NO:25) (FIG. 8B) and Tat(47-57) (SEQ ID NO:26) (FIG. 8C). Cells were incubated with the indicated peptide conjugated to the pro-apoptotic peptide (klaklak)2 for 16 hr. Cell viability was then assessed using MTT assay. The Y axis represents the % of viable cells (normalized by untreated cells). The X axis represents the log uM concentration. The results are an average of duplicates. As shown in FIG. 8, the conjugation of (klaklak)2 to the CPPs; R9 or Tat(47-57), resulted in an effective dose response curve, indicated by a strong effect of (klaklak)2 peptide on cell apoptosis with the internalization mediated by the CPPs, where conjugation to a non penetrating peptide, N2 resulted in a flat line (i.e. no dose response curve—no effect).

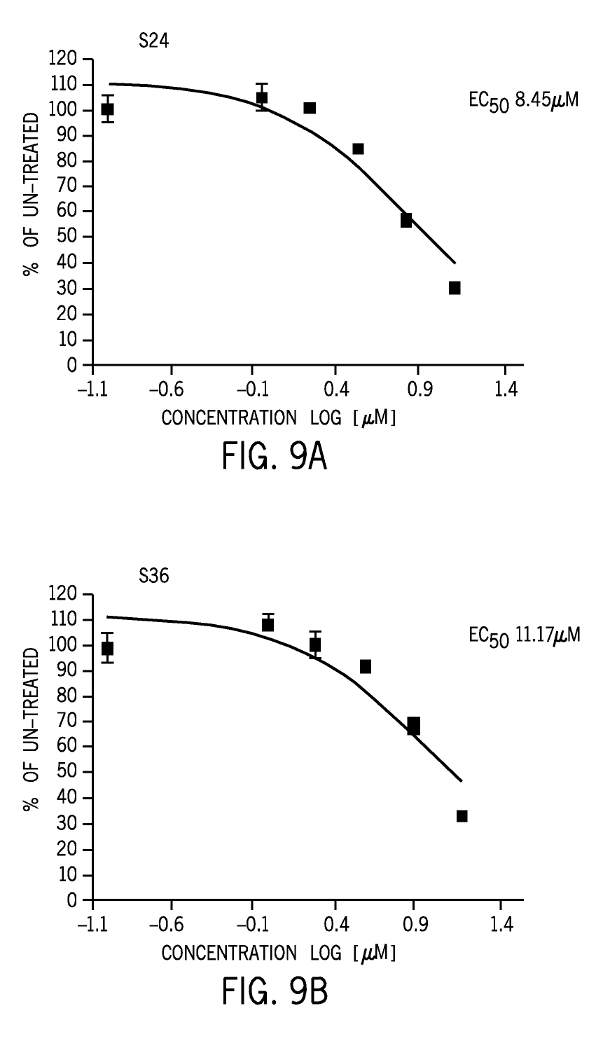

FIG. 9 demonstrates the results of the viability of Hela cells incubated with the pro-apoptotic peptide conjugated to S24 (SEQ ID NO: 27) (FIG. 9A) and S36 (SEQ ID NO: 29) (FIG. 9B). Cells were incubated with the indicated peptide conjugated to the pro-apoptotic peptide (klaklak)2 for 16 hr. Cell viability was then assessed using MTT assay. The Y axis represents the % of viable cells normalized by untreated cells of duplicated experiments. The X axis represents the log uM concentration. As shown in FIG. 9, both S24 (SEQ ID:27) and S36 (SEQ ID:29) are able to efficiently deliver (klaklak)2 into the cell in dose dependent manner with an EC50 of 8.45 and 11.17 respectively.

### Example 4

### In Vitro Cytotoxicity Measurements

In order to assess the in vitro cytotoxicity of the peptides, an LDH (lactate dehydrogenase) analysis was carried out, using LDH kit (Promega G1782), according to manufacturer instructions.

Briefly, HeLa cells from 5 ml of confluent T75 flask were seed in 96 well plate at 10×10⁴ cells/mL in 0.1 ml DMEM supplied with 10% Fetal Calf Serum. After 24 hrs incubation with growth medium, wells were replaced with Serum Free growth medium for 24 hrs, and then cells were washed with OptiMEM and replaced with 0.1 ml peptide solution at 10 uM for 1 h at 37° C. 50 ul of supernatant were transferred to the new plate and detected according to Cyto Tox 96, Non Radioactive cytotoxicity assay, (LDH kit Promega G1782). Table 5 presents the results of in vitro cytotoxicity measurements by LDH for S22, S24, S36 and S40 peptides and two positive control peptides; Tat(47-57) and R9. LDH experiment was done with fluorescent labeled (5-FAM) peptides in concentration of 10 ug/ml and the % of cytotoxicity was measured by LDH levels. As shown in Table 5, all peptides maintain low levels of cytotoxicity.

TABLE 5

| SEQ ID NO | Peptide name | LDH % cytotoxicity |
|---|---|---|
| 19 | Tat(47-57) | 2 |
| 21 | R9 | 2 |
| 1 | S22 | 7 |
| 2 | S24 | 10 |
| 3 | S36 | 3 |
| 4 | S40 | 8 |

### Example 5

### Nano-Sized Particle Cargo Selective Delivery by CPPs

In order to examine CPPs capability to selectively target sarcomas and other soft tissue cancers and to deliver particle into cancer cells, nano-sized particles made from natural lipids with hyaluronic acid coating were conjugated to CPPs and binding and internalization were examined on NRK and A549 cell lines.

Liposomes Preparation:

Liposome preparation was carried out as described in Peer D., at al., 2008, Science 319, 627-630, Briefly, liposomes (60% soy-pc, 20% DPPE 20% cholesterol mol/mol) were prepared at a concentration of 40 mg/ml (Soy PC-273 mg, DPPE-81.2 mg Cholesterol-145.4 mg in 10 ml of ethanol). After a complete dissolvent, the solution was evaporated in a rotary evaporator (BUCHI R-210). Following the evaporation the dry lipid film was hydrated in 10 ml of HEPES (PH 7.4) and the solution was shaken (2 hr 65° C.). The MLV were reduced to ULV with an average size of ˜150 nm (Zetasizer Nano ZS system) by passing them through an extruder (Lipex) using filtration membranes in a serially manner (0.4 nm, 0.2 nm, 0.1 nm). Average zeta potential of the ULV was −3 mV (Zetasizer Nano ZS system).

Hyaluronan Coating:

Hyaluronan (HA) (700 KDa Lifecore) was dissolved in 0.2M MES buffer (PH 5.5) to a final concentration of 5 mg/ml. HA was activate with EDC and solfo-NHS at a molar ratio of 1:1:6. After 30 min of activation the liposomes were added and the pH was adjusted to 7.4. The solution was incubated at room temp (2 hr). The free HA was removed by 3 repeated ultracentrifugations. The resulting HA-ULV's were at an average of 175 nm in size.

Cell Penetrating Peptide Binding and Purification:

CPPs S22 (SEQ ID NO: 1), S24 (SEQ ID NO: 2) and S8 (SEQ ID NO: 6) and Control CPPs; 9R with additional C-terminal Lysine, 9R-K (SEQ ID NO: 40) and N2 (SEQ ID NO: 22), were dissolved to a concentration of 1 mg/ml. HA-ULV's (250 μl of 40 mg/ml) were activated with 1 ml of 0.4M EDC and 1 ml of 0.4 mM solfo-NHS. After 30 min the activated liposomes were divided into 5 test tubes (250 μl per each tube) and a different CPP (50 μl) was added. After incubation at RT for 2 hr, the CPP-HA-ULVS were dialyzed over night with PBS buffer (0.5 L/3 buffer changeovers) to remove unbound CPP, using dialysis bags with a cutoff 3500 Da.

Fluorescence Activated Cell Sorting (FACS):

For binding analysis, 3.5×105 cells were trypsinized, spin down, re-suspended with FACS buffer (1% fetal bovine serum in 1×PBS), incubated for 30 minutes on ice with different CPPs (Conjugated to FAM fluorophore) and washed with 1 ml FACS buffer.

Internalization Assay:

Internalization assay was performed in 24 well plates. A549 or NRK cells were seeded on cover slips in RPMI or DMEM medium respectively, supplemented with antibiotics, L-Glutamine and 10% fetal calf serum (Biological industries, Beit Haemek).

For membrane staining, cells were stained with CellTracker™ DilC18 (5)-DS solution (Invitrogen, Carlsbad, Calif., USA), diluted 1:5000 with PBS. For internalization experiments involve NPs Concanavalin A, Alexa fluor 647 conjugate (10 μg/ml) (C21421, Invitrogen,) was used to label cell membrane. For nuclei staining, cells were stained with Hoechst (1:10,000 in PBS) (33258, Sigma). Cells were exposed to CPPs only (10 μM) or CPP-liposome complex (50 μl from stock, according to preparation method) or liposome only (50 μl from the prepared liposomal stock solution) in medium without serum for a period of 1 hour at 37° C. in a humidified atmosphere with 5% CO2. Subsequently, the cells were washed twice using cold PBS, fixated with 4% Paraformaldehyde (PFA) and washed again with cold PBS. Membrane and nuclei staining were preformed after fixation. The cells were mounted by fluorescent mounting medium (Golden Bridge international, Mukilteo, Wash., USA) and fluorescent was measured using Andor Spinning disc confocal microscope and the Meta 510 Zeiss LSM confocal microscope. Laser beams at 405, 488, 561 and 650 nm were used for UV, FAM, Rhodamine, Concavaline A and CellTracker™, fluorophores excitation respectively. Serial optical sections of the cells were recorded for each treatment and the images were processed using Zeiss LSM Image browser software.

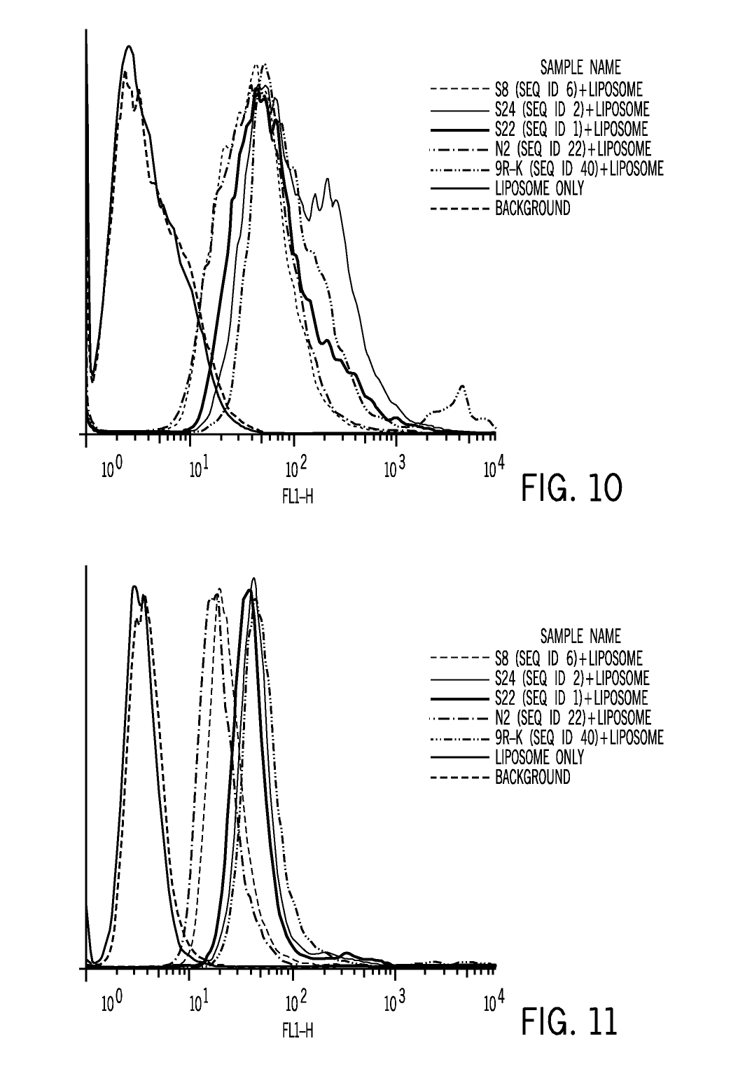

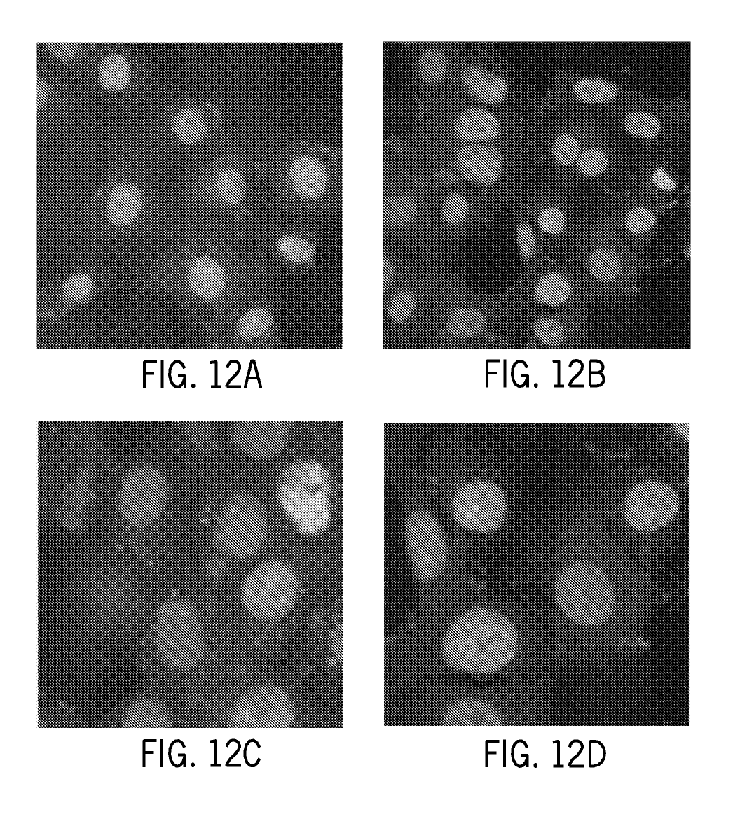

The Binding capacity of each of the five CPPs, two controls and 3 CPPs (respectively SEQ ID NOs: 40, 22 and 1, 2 and 6) conjugated liposomes to NRK and A549 cell lines was tested using FACS analysis (FIGS. 10 and 11 respectively). According to FACS analysis, CPPs numbers 9R-K (SEQ ID NO: 40), S22 (SEQ ID NO: 1) and S24 (SEQ ID NO: 2) conjugated to liposomes showed high binding levels to A549 cells (FIG. 11), while CPPs N2 (SEQ ID NO: 2) and S8 (SEQ ID NO: 6) showed lower binding effect (FIG. 11). CPP S24 (SEQ ID NO: 2) showed the strongest binding effect to NRK cells (FIG. 10).

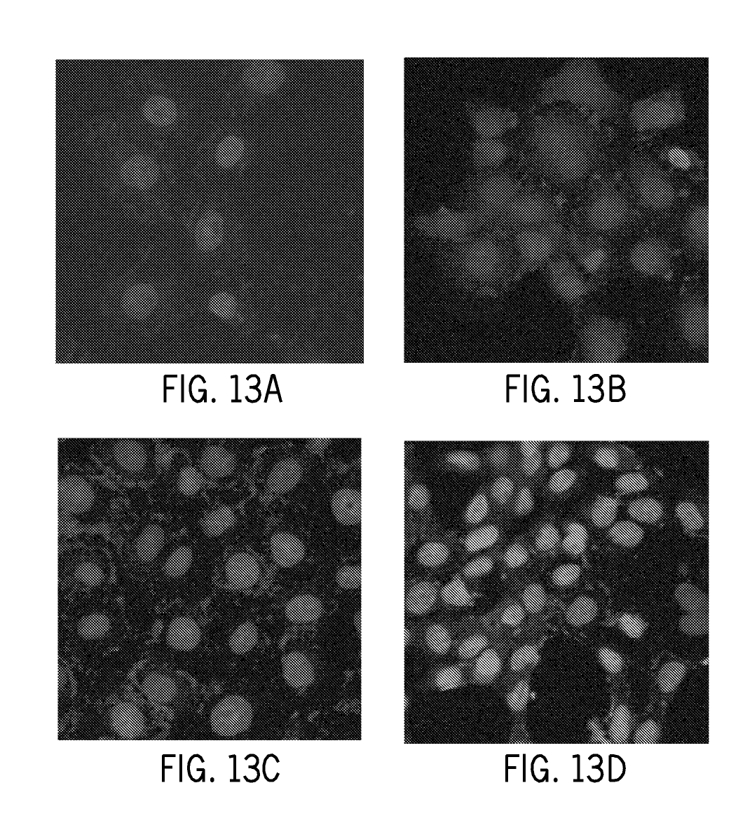

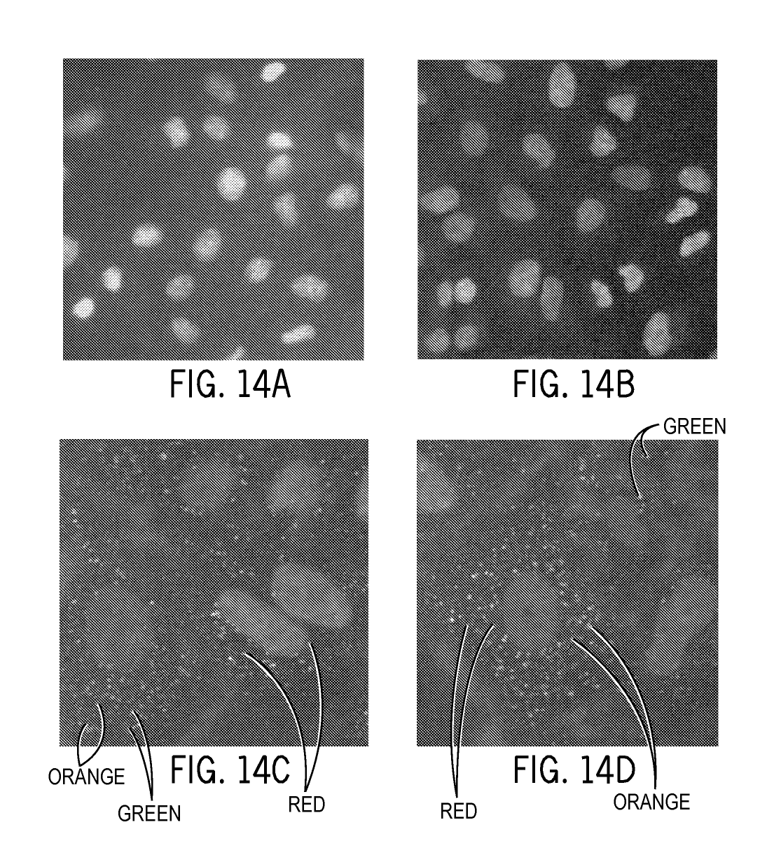

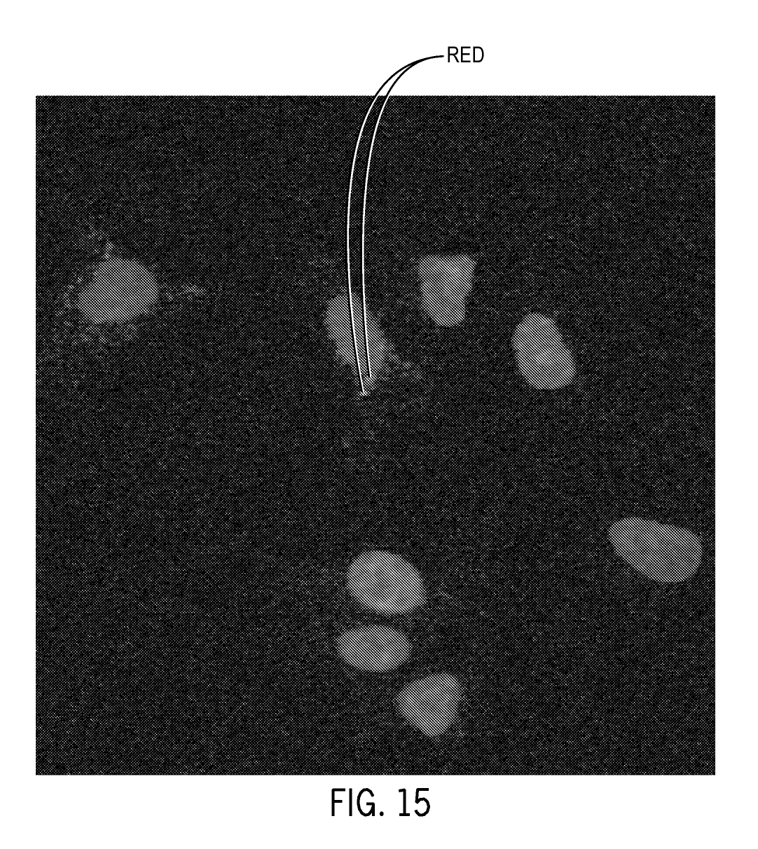

Internalization tests were performed using confocal microscopy. CPPs conjugated to liposomes were tested for internalization into NRK and A549 cell lines. The results are presented in FIGS. 12-15. Liposome conjugated CPPs S22 (SEQ ID NO: 1) and S8 (SEC) ID NO: 6) showed a weak internalization into NRK cell line (FIGS. 12 and 13, respectively). However liposome conjugated CPP S22 (SEQ NO: 1) showed a better penetration into NRK in a comparison with peptide only (FIG. 12) and with liposome only. CPP N2 (SEQ ID NO: 22) did not show internalization effect in A549, as expected from a negative control peptide. CPP S22 (SEQ ID NO: 1) showed a significant internalization effect into A549 cells (FIG. 14) in a comparison with peptide only and with liposome only (FIG. 15). The internalization effect of 9R-K (SEQ ID NO: 40) and S24 (SEQ ID NO: 2) was not evaluated due to non specific binding of those peptides to cell membrane (data not shown).

### Example 6

### Kinetic Profiling of CPP Peptides Using Image Based High Content Screening

Image-based High Content Analysis (HCA) is an automated imaging approach for high throughput microscopic analysis, conceived to deeply understand cellular phenotypes. This technology allows measuring spatial distribution, morphology and staining intensity of targets within cells, organelles, or whole cells. Therefore, HCS allows the description of complex cellular (or multicellular) phenotypes. In addition, it provides the flexibility to measure cell subpopulations and to combine multiple measurements per cell, while simultaneously eliminating unwanted cells and artifacts. As a consequence of these abilities, HCA provides the most effective way for studying, with high throughput and at the maximal level of sub-cellular detail, the effect of compounds/treatments/conditions over the cell, thus allowing the use of this technology for lead discovery.

HCA involves probing cells with fluorescent markers, capturing images of the cells very rapidly with high-resolution image instrumentation and extracting detailed information from the images with powerful high content imaging software. This software is able to segment each different compartment (according to pixel level of intensity) within a cell that has been traced by a given fluorescent marker, allowing the quantification of numerous parameters for each of the segmented compartments and the relation between these sub-cellular compartments. Thus, the effect of a given molecule or drug carrier on the intrinsic cell biology can be intensively investigated.

Unlike other high throughput systems not based on cell images, HCS enables the area measurements of each stained compartment or sub-compartment for each cell in the population to be separately determined. Thus, since HCS is capable of analyzing the whole cell population within a reasonable timeframe, HCS is generally considered to be less biased than conventional low throughput microscopy (Ramirez., et al., ASSAY and Drug Development Technologies. June 2011, 9(3): 247-261; Assessing cellular toxicities in fibroblasts upon exposure to lipid-based nanoparticles: a high content analysis approach. Leonardo J Solmesky et al.)

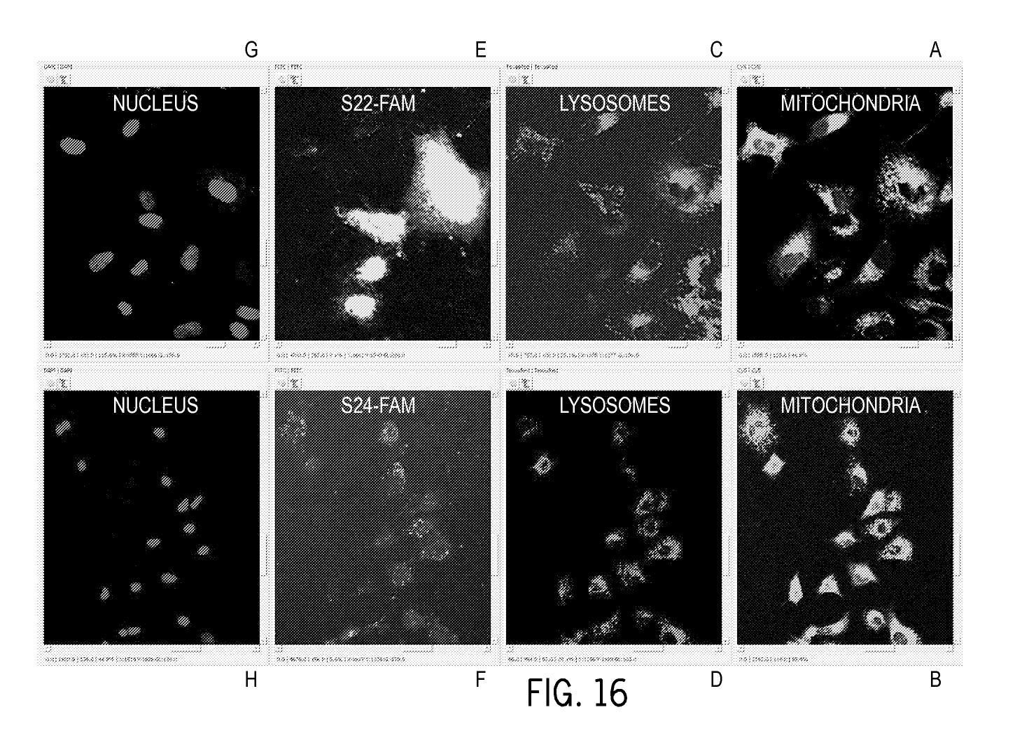

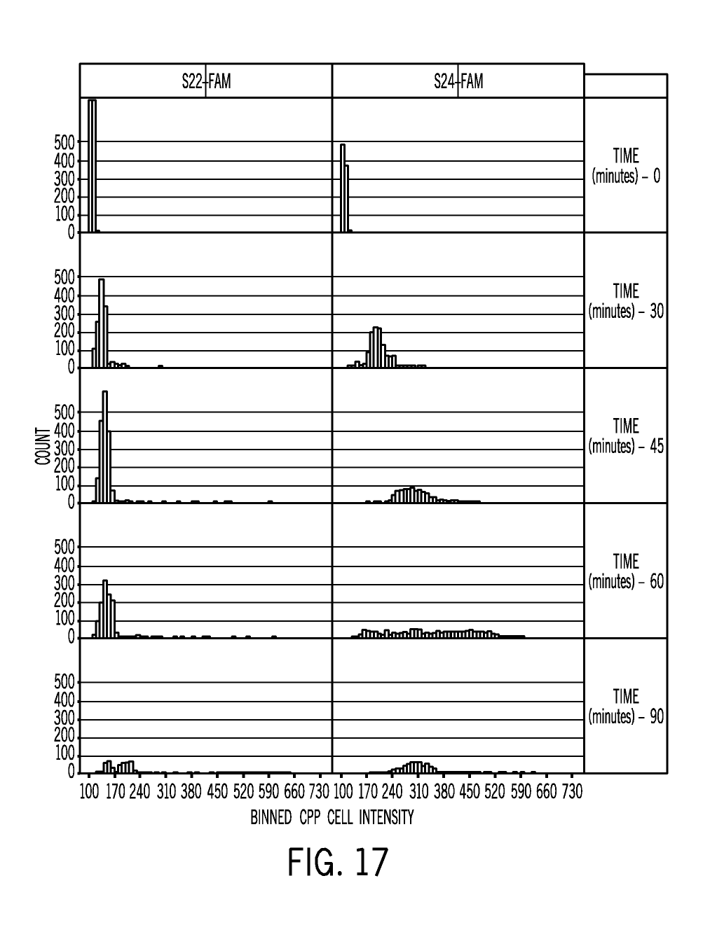

In this example HCS was utilized in order to investigate the kinetics of the biological functionality of two CPP peptides, S22 (SEQ ID NO 1) and S24 (SEQ ID NO 2), through binding to the A549 cell line. The following protocol was used: Greiner μclear 96 well plates (Microplates Greiner u Clear 96w, TC, sterile, black, lid, 32/case costs, Degroot) were coated with PDL (Poly-D-lysine), followed by seeding of 10000 A549 cells/well and incubation for 24 hours at 37° C. Then the plates were washed with DMEM (DMem (Hg) W/O Na Pyr (Ce)), and CPP peptides were added at different times. Following additional washing with PBS (phosphate buffered saline), HBSS (Hanks' balanced salt solution) and staining (nucleus, ER, Mitochondria), as shown in FIG. 16. In the next step images were acquired, followed by software analysis; the results are visualized as shown in FIG. 17.

FIG. 16 presents a snapshot image of a stained subpopulation using HCA technology. Four different colors enable visualization of the Nucleus, Penetration of CPP, Lysosome and mitochondria. This figure demonstrates the coherency in the staining of each element with its distinguished color. Images were taken using In Cell Analyzer 2000 (GE Healthcare, UK).

FIG. 17 shows results of kinetic profiling of CPPs: S22 and S24 (SEQ ID NO 1 and 2, respectively) on A549 cell line. A histogram of the CPPs labeling intensity of cell population versus time is presented. Two histograms (S22 and S24) are presented for each time line, beginning from time zero (upper two histograms) and terminating with 90 minutes (bottom two histograms). It is shown that for both peptides, distribution of cell labeling intensity, representing the amount of successfully penetrated peptides, progresses to the right, thus demonstrating time dependent internalization with saturation time point between 60 to 90 minutes. Analysis of the derivatives of the average of these histograms indicated that S24 demonstrates a faster kinetic profile than S22 in this experiment. Histograms were generated using the “In cell investigator 1.6” software on images acquired by the In Cell Analyzer 2000 device (GE Healthcare, UK).

It will be appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination. It will also be appreciated by persons skilled in the art that the present invention is not limited by what has been particularly shown and described hereinabove. Rather the scope of the invention is defined only by the claims which follow.

1. A peptide consisting essentially of an amino acid sequence set forth in any one of SEQ ID NOs: 1-12 or 33, or a variant thereof.

2. (canceled)

3. The peptide according to claim 1, wherein:
the peptide comprises from 5 to at most 33 consecutive amino acid residues of the peptide set forth in SEQ ID NO: 7;
the peptide comprises from 5 to at most 50 consecutive amino acid residues of the peptide set forth in SEQ ID NO: 33;
the peptide comprises from 5 to at most 31 consecutive amino acid residues of the peptide set forth in SEQ ID NO: 9;
the peptide comprises from 5 to at most 32 consecutive amino acid residues of the peptide set forth in SEQ ID NO: 10;
the peptide comprises from 5 to at most 31 consecutive amino acid residues of the peptide set forth in SEQ ID NO: 11; or
the peptide comprises from 5 to at most 34 consecutive amino acid residues of the peptide set forth in SEQ ID NO: 12.

4. The peptide according to claim 3, wherein:
the peptide comprises from 9 to 19 consecutive amino acid residues of the peptide set forth in SEQ ID NO: 7;
the peptide comprises from 9 to 19 consecutive amino acid residues of the peptide set forth in SEQ ID NO: 8;
the peptide comprises from 6 to 16 consecutive amino acid residues of the peptide set forth in SEQ ID NO: 9;
the peptide comprises from 7 to 17 consecutive amino acid residues of the peptide set forth in SEQ ID NO: 10;
the peptide comprises from 6 to 16 consecutive amino acid residues of the peptide set forth in SEQ ID NO: 11; or
the peptide comprises from 9 to 19 consecutive amino acid residues of the peptide set forth in SEQ ID NO: 12.

5. (canceled)

6. (canceled)

7. The peptide according to claim 1, wherein the peptide is selected from the group comprising: a peptide having an amino acid sequence consisting essentially of the amino acid sequence KKTCRRINYCALNK (SEQ. ID. No. 1);
a peptide having an amino acid sequence consisting essentially of the amino acid sequence KKTLKKLWKKKKRK (SEQ. ID. No. 2);
a peptide having an amino acid sequence consisting essentially of the amino acid sequence RRRHQRKYRRY (SEQ. ID. No. 3);
a peptide having an amino acid sequence consisting essentially of the amino acid sequence FLRRRRRFTRQT (SEQ. ID. No. 4),
a peptide having an amino acid sequence consisting essentially of the amino acid sequence KKLALYLLLAL (SEQ. ID. No. 5),
a peptide having an amino acid sequence consisting essentially of the amino acid sequence KPSARMLLLKGFGK (SEQ. ID. No. 6),
and a derivative thereof.

8. The peptide of claim 7, further comprising one or more of the following modifications: glycosylation, amidation, acetylation, acylation, alkylation, alkenylation, alkynylation, phosphorylation, sulphorization, hydroxylation, hydrogenation, cyclization, ADP-ribosylation, anchor formation, covalent attachment of a lipid or lipid derivative, methylation, myristylation, pegylation, prenylation, or ubiquitination, or a combination thereof.

9. The peptide of claim 7, further comprising a linkage group selected from the group comprising thioethers, wherein the linkage group replaces the disulfide bond formed by a Cysteine amino acid residue of the peptide.

10. The peptide of claim 7, further comprising a non-natural amino acid as listed in Table 1 or a non-natural amino acid selected from the group consisting of non-natural amino acids include beta-amino acids (beta3 and beta2), homo-amino acids, cyclic amino acids, aromatic amino acids, Pro and Pyr derivatives, 3-substituted Alanine derivatives, Glycine derivatives, ring-substituted Phe and Tyr Derivatives, linear core amino acids and diamino acids; or a combination thereof.

11. (canceled)

12. The peptide of claim 7, wherein the peptide comprises at least one of: C-terminal amidation; N-terminal acetylation; PEGylation; folate addition.

13. The peptide according to claim 12, wherein the peptide is amidated at its C-terminus and acetylated at its N-terminus.

14. The peptide according to claim 7, wherein the peptide further comprises a moiety which is suitable for detection using a method for detection, wherein such moiety is selected from the group comprising fluorophores, radioactive tracers and haptens.

15. The peptide according to claim 14, wherein the peptide is radioactively labeled, optionally by having incorporated a radioactively labeled amino acid.

16. The peptide according to claim 14, wherein the hapten comprises biotin.

17. A complex or a fusion molecule or a conjugate comprising a peptide according to claim 7 and a cargo molecule, wherein the cargo molecule is selected from the group comprising nucleic acid molecules, amino acids, therapeutically active peptides, proteins, carbohydrates, lipids, a contrast or imaging agent, a quantum dot, a diagnostic agent and a therapeutically active molecule having a molecular weight of 1000 Daltons or less, and a combination thereof.

18. (canceled)

19. The complex or a fusion molecule or a conjugate according to claim 17, wherein the cargo molecule is covalently or non-covalently bound to the peptide.

20. (canceled)

21. The complex or a fusion molecule or a conjugate according to claim 17, wherein the cargo molecule is present within or as part of a structure, wherein the structure is selected from the group consisting of nanoparticles, microparticles, liposomes, nanotubes and micelles.

22. The complex or a fusion molecule or a conjugate according to claim 17, wherein the nucleic acid molecule is selected from the group comprising nucleic acids, DNA molecules, RNA molecules, PNA molecules, siRNA molecules, miRNA molecules, antisense molecules, ribozymes, aptamers, spiegelmers and decoy molecules.

23. The complex or a fusion molecule or a conjugate according to claim 22, wherein the nucleic acid molecule is a nucleic acid-based vaccine.

24. The complex or a fusion molecule or a conjugate according to claim 17, wherein the cargo molecule is a therapeutically active peptide comprising an antigen suitable for vaccination.

25. (canceled)

26. (canceled)

27. (canceled)

28. (canceled)

29. (canceled)

30. (canceled)

31. A method of using the peptide according to claim 1 as a cell-penetrating peptide for treatment of cancer, comprising administering the peptide to a subject in need of treatment thereof.

32. The method of claim 31, wherein the subject has a tumor, the method further comprising specifically targeting the tumor with the peptide.

33. The method of claim 32, wherein said administering the peptide comprises administering the peptide coupled via a protease cleavable linker to a neutralizing peptide to inhibit cell penetration of the peptide, the peptide targeting the tumor; the method further comprising exposing the protease cleavable linker to a tumor protease, cleaving the protease cleavable linker to release the peptide and penetrating tumor cells by the peptide.

34. The method of claim 33, further comprising imaging the tumor according to said penetrating tumor cells by the peptide.

35. A method of using the complex, fusion molecule or conjugate according to claim 17 for treatment of cancer, comprising administering the complex, fusion molecule or conjugate to a subject in need of treatment for cancer.

36. The method of claim 35, wherein the subject has a tumor, the method further comprising specifically targeting the tumor with the peptide.

37. The method of claim 36, wherein said administering the complex, fusion molecule or conjugate comprises administering the complex, fusion molecule or conjugate, wherein the peptide is coupled via a protease cleavable linker to a neutralizing peptide to inhibit cell penetration of the peptide, the peptide targeting the tumor; the method further comprising exposing the protease cleavable linker to a tumor protease, cleaving the protease cleavable linker to release the complex, fusion molecule or conjugate and penetrating tumor cells by the complex, fusion molecule or conjugate.

38. The method of claim 37, further comprising imaging the tumor according to said penetrating tumor cells by the complex, fusion molecule or conjugate.
